(19) United States
(12) Patent Application Publication
(10) Pub. No.: US 2009/0069324 A1
(43) Pub. Date: Mar. 12, 2009

(54) CHEMICAL INHIBITORS OF BFL-1 AND RELATED METHODS

(75) Inventors: John C. Reed, Rancho Santa Fe, CA (US); Dayong Zhai, San Diego, CA (US); Shinichi Kitada, San Diego, CA (US); Eduard Sergienko, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: The Burnham Institute, La Jolla, CA (US)

(21) Appl. No.: 12/130,735

(22) Filed: May 30, 2008

Related U.S. Application Data
(60) Provisional application No. 60/934,843, filed on Jun. 14, 2007.
(60) Provisional application No. 61/023,372, filed on Jan. 24, 2008.
(60) Provisional application No. 61/039,558, filed on Mar. 26, 2008.

(51) Int. Cl. A61K 31/496; A61K 31/5377; G01N 33/53; A61P 35/00

(52) U.S. Cl. 514/235.5; 514/254.01; 436/501

(57) ABSTRACT

Compounds that bind to Bfl-1 as well as conjugates of such compounds are provided. Various embodiments additionally provide methods of using such compounds to identify additional anti-apoptotic Bfl-1 binding compounds. Methods of using such compounds to increase apoptosis in a cell are also provided.

### RELATED APPLICATIONS

This application claims priority under 35 U.S.C. §119(e) to U.S. Provisional Application No. 60/934,843, filed Jun. 14, 2007, U.S. Provisional Application No. 61/023,372, filed Jan. 24, 2008, and U.S. Provisional Application No. 61/039,558, filed Mar. 26, 2008, each of which is incorporated herein by reference in its entirety.

### STATEMENT REGARDING FEDERALLY SPONSORED R&D

This invention was made with government support under Grant Number: CA113318 awarded by the National Institutes of Health, and Grant Number: 1 X01 MH077632-01 awarded by the National Institutes of Health. The government has certain rights in the invention.

### BACKGROUND

1. Field

The present invention relates generally to molecular medicine and research, and more specifically to compositions and methods for altering cell death regulatory molecules.

2. Description of the Related Art

Normal tissues in the body are formed either by cells that have reached a terminally differentiated state and no longer divide or by cells that die after a period of time and are replaced from a pool of dividing cells. For example, nervous tissue is formed early in development, and the cells of the nervous system reach a terminally differentiated state soon after birth. In contrast, the body has a number of self renewing tissues such as skin, gut, bone marrow and sex organs that undergo a balanced flux of cell birth and death. This flux, which results in the production of 50-70 billion cells per day in an average adult and amounting to a mass of cells equivalent to an entire body weight over a years' time, is balanced by the regulated eradication of an equivalent number of cells. In self renewing tissues, the eradication is maintained, in part, due to the process of programmed cell death, known as apoptosis, in which the cells are genetically “programmed” to die after a certain period of time.

Apoptosis is particularly prominent during the development of an organism, where cells that perform transitory functions are programmed to die after their function no longer is required. In addition, apoptosis can occur in cells that have undergone major genetic alterations, thus providing the organism with a means to rid itself of defective and potentially cancer forming cells. Apoptosis also can be induced due to exposure of an organism to various external stimuli, including, for example, bacterial toxins, ethanol and ultraviolet radiation. Chemotherapeutic agents for treating cancer also are potent inducers of apoptosis.

The regulation of programmed cell death is a complex process involving numerous pathways. On occasion, defects occur in the regulation of programmed cell death. Given the critical role of this process in maintaining a steady-state number of cells in a tissue or in maintaining the appropriate cells during development of an organism, defects in programmed cell death often are associated with pathological conditions. It is estimated that either too little or too much cell death is involved in over half of the diseases for which adequate therapies do not currently exist.

Various disease states occur due to aberrant regulation of programmed cell death in an organism. For example, defects that result in a decreased level of apoptosis in a tissue as compared to the normal level required to maintain the steady-state of the tissue can result in an increased number of cells in the tissue. Such a mechanism of increasing cell numbers has been identified in various cancers, where the formation of a tumor occurs not because the cancer cells necessarily are dividing more rapidly than their normal counterparts, but because the cells are not dying at their normal rate.

Thus, a need exists for agents capable of modulating programmed cell death pathways and methods for treating individuals experiencing diseases associated with aberrant regulation of programmed cell death. The present invention satisfies this need and provides additional advantages as well.

### SUMMARY

Some embodiments of the invention provide compounds that bind to anti-apoptotic Bcl-2 polypeptides as well as conjugates of such compounds. Another embodiment of the invention additionally provides methods of using such compounds to identify additional anti-apoptotic Bcl-2 polypeptide binding compounds. Another embodiment of the invention further provides methods of using such compounds to increase apoptosis in a cell.

A method of modulating the activity of Bfl-1 is disclosed in accordance with some embodiments of the present invention. In some embodiments, the method comprises contacting a Bfl-1 polypeptide with a small molecule. In some embodiments, the small molecule can be selected from the group of molecules shown in Tables 2-8.

A method of inhibiting binding of a Bfl-1 polypeptide to a BH3 domain is disclosed in accordance with some embodiments of the present invention. In some embodiments, the method comprises contacting a Bfl-1 polypeptide with a compound having a core structure selected from core structures I, II, III and IV or a compound selected from the compounds shown in Tables 2-8, thereby inhibiting binding of said Bfl-1 polypeptide to a BH3 domain.

A method of increasing apoptosis in a cell is disclosed in accordance with some embodiments of the present invention. In some embodiments, the method comprises contacting a cell with an effective amount of a compound having a core structure selected from core structures I, II, III and IV or a compound selected from the compounds shown in Tables 2-8, whereby binding of a Bfl-1 polypeptide to a BH3 domain is inhibited and apoptosis is increased.

A method of reducing the severity of a pathological condition in an individual is disclosed in accordance with some embodiments of the present invention. In some embodiments, the method comprises administering to an individual having a pathological condition characterized by a pathologically reduced level of apoptosis a compound having a core structure selected from core structures I, II, III and IV or a compound selected from the compounds shown in Tables 2-8, whereby binding of a Bfl-1 polypeptide to a BH3 domain is inhibited and the severity of said pathological condition is reduced. In some embodiments, the pathological condition is cancer, psoriasis, hyperplasia, an autoimmune disease, an inflammation-associated disease or restenosis.

A method of identifying a Bfl-1 binding compound is disclosed in accordance with some embodiments of the present invention. In some embodiments, the method comprises: contacting a Bfl-1 polypeptide with a candidate compound in the presence of a compound labeled with a detectable moiety, wherein the labeled compound is selected from a compound having a core structure selected from core structures I, II, III and IV or the compounds shown in Tables 2-8; and measuring the binding of the labeled compound to the Bfl-1 polypeptide, wherein a decrease in binding of the labeled compound in the presence of the candidate compound relative to the absence of the candidate compound identifies a Bfl-1 binding compound. In some embodiments, the detectable moiety is a fluorophore, chromophore, paramagnetic spin label, radionucleotide, or binding group having specificity for another molecule that can be detected. In some embodiments, the Bfl-1 polypeptide lacks a C-terminal hydrophobic transmembrane domain.

A conjugate comprising a compound selected from a compound having a core structure selected from core structures I, II, III and IV or the compounds shown in Tables 2-8 conjugated to a detectable moiety is disclosed in accordance with some embodiments of the present invention.

A complex comprising a Bfl-1 polypeptide bound to a compound having a core structure selected from core structures I, II, III and IV or a compound selected from the compounds shown in Tables 2-8 is disclosed in accordance with some embodiments of the present invention.

A method of screening for compounds capable modulating the activity of Bfl-1 is disclosed in accordance with some embodiments of the present invention. In some embodiments, the method comprises: providing a Bfl-1 polypeptide; providing a fluorescently labeled compound known to bind Bfl-1; and contacting the Bfl-1 polypeptide and the binding compound in the presence or absence of a candidate binding compound or library of candidate binding compounds; and determining fluorescence of the Bfl-1 polypeptide, wherein a decrease in fluorescence indicates that the candidate binding compound inhibits binding of the binding compound to the Bfl-1 polypeptide.

In some embodiments, the candidate binding compound can be a natural product or natural product derivative. In some embodiments, the fluorescent label can be selected from the group consisting of Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red. In some embodiments, the compound known to bind to Bfl-1 can be a peptide, peptide analog or a small molecule. In some embodiments, the peptide can be Bid-BH3 peptide. In some embodiments, the method can further comprise at least one secondary screen to confirm that said candidate binding compound modulates the activity of Bfl-1. In some embodiments, the secondary screen can be an apoptosis assay. In some embodiments, screening method can be in high throughput format. In some embodiments, the fluorescence can be measured by fluorescence polarization. In some embodiments, the fluorescence can be measured by time-resolved fluorescence resonance energy transfer (TR-FRET), solid phase amplification (SPA) or an ELISA-like assay. In some embodiments, the decrease in fluorescence can be at least 20, 30, 40, or 50%.

A method of optimizing a target compound is disclosed in accordance with some embodiments of the present invention. In some embodiments, the method comprises: providing a Bfl-1 polypeptide; providing a fluorescently labeled compound known to bind to the Bfl-1 polypeptide; contacting the Bfl-1 polypeptide and the fluorescently labeled compound in the presence or absence of a test compound or library of test compounds; determining fluorescence of the Bfl-1 polypeptide, wherein a decrease in fluorescence indicates that the test compound inhibits binding of the fluorescently labeled compound to the Bfl-1 polypeptide; determining whether the test compound binds to the Bfl-1 polypeptide at a position adjacent to a chemical fragment; and linking the chemical fragment to the test compound If the chemical fragment binds adjacent the test compound. In some embodiments, the chemical fragment can be covalently linked to the test compound.

### BRIEF DESCRIPTION OF THE DRAWINGS

FIG. 1 shows purification of GST-Bcl-2 proteins.

FIG. 2 shows fluorescence polarization assay (FPA) analysis of Bcl-2-family proteins using fluorescein isothiocyanate (FITC)-Bid BH3 peptide.

FIGS. 3A-3G show competition assay analysis of green tea compound EGCG.

FIG. 4 shows Z′factor determination for Bfl-1 FPA.

FIG. 5 shows an example of screening results.

FIG. 6A shows MLS-0067130 Microsomal Stability. FIG. 6B depicts MLS-0067130 Plasma Stability.

FIGS. 7A-7D show selectivity of Bfl-1 antagonists.

FIG. 8 shows CID-779754 non-toxicity.

FIGS. 9A and 9B show induction of apoptosis by MLS-0009480 (A) and MLS-0051609 (B). “C” indicates the DMSO (solvent only) control.

FIGS. 10A and 10B show induction of apoptosis by MLS-0047123 (A) and MLS-0051509 (B). “C” indicates the DMSO (solvent only) control.

### DETAILED DESCRIPTION

Various embodiments disclosed herein are generally directed towards compositions and methods for inhibiting Bcl-2 polypeptides. Compounds and methods of using such compounds are providing to inhibit anti-apoptotic Bcl-2 polypeptides such as, for example, the Bcl-2 polypeptide Bfl-1. The compounds can be used to inhibit the anti-apoptotic activity of Bcl-2 family polypeptides, for example, by mimicking the effects of BH3 domain-containing polypeptides. In some embodiments, the compounds can be used, for example, to increase or promote apoptosis in a cell. Therefore, the compounds and methods of various embodiments of the invention can be used, for example, to reduce the severity of a pathological condition characterized by a pathologically reduced level of apoptosis, for example, to treat a pathological condition such as cancer or autoimmune diseases. In other embodiments, the compounds disclose herein can be used as research probes for Bcl-2 polypeptides such as, for example, Bfl-1.

The present invention also provides methods for identifying compounds that bind to a Bcl-2 polypeptide, and neutralizes the anti-apoptotic activity of these polypeptides. Compounds identified using the screening methods described herein that bind to Bcl-2 polypeptides and alter their function in apoptosis are also provided. These compounds include “lead” peptide compounds and “derivative” compounds constructed so as to have the same or similar molecular structure or shape as the lead compounds but that differ from the lead compounds either with respect to susceptibility to hydrolysis or proteolysis and/or with respect to other biological properties, such as increased affinity for the receptor.

Apoptosis is governed in part by Bcl-2-family proteins. The human genome contains six genes that encode anti-apoptotic members of the Bcl-2 family, Bcl-2, Bcl-XL, Mcl-1, Bcl-W, Bfl-1 and Bcl-B (Reed et al., Sci. STKE re9 (2004); Reed and Pellecchia, Blood 106:408-418 (2005), each of which is incorporated herein by reference). Among these is Bfl-1, also known as A1 in mice, an NF-κB-inducible member of the Bcl-2 family. The human genome contains six genes that encode anti-apoptotic members of the Bcl-2 family of which Bfl-1 is a member (Oltersdorf et al., Nature 435, 677-681 (2005); Palmer et al., Proc Natl Acad Sci USA 101, 17404-17409 (2004). Other Bcl-2-family anti-apoptotic proteins include Bcl-2, Bcl-XL, Mcl-1, Bcl-W, and Bcl-B. The mouse ortholog of Bfl-1 has four gene copies (A1a-d). Bfl-1 interacts with other Bcl-2 family members, such as the pro-apoptotic BH3-interacting domain death agonist (BID). Bfl-1 is highly expressed in lymphoid tissues. Therefore, compounds that inhibit anti-apoptotic Bcl-2 polypeptides such as Bfl-1 can be used to promote apoptosis and to treat a pathological condition having a reduced level of apoptosis, particularly those involving lymphoid tissues and inflammatory cells. Compounds that inhibit binding of anti-apoptotic Bcl-2 polypeptides to BH3 domain-containing pro-apoptotic proteins can be used to stimulate apoptosis in a cell.

One embodiment of the invention provides compounds that bind to an anti-apoptotic Bcl-2 polypeptide, including agonists and antagonists of anti-apoptotic Bcl-2 polypeptide activity. For example, one embodiment of the invention provides a compound as shown in any of Tables 2-8. As disclosed herein, various core structural motifs were identified among the compounds found to bind to an anti-apoptotic Bcl-2 polypeptide. For example, a sulfonyl pyrimidine core scaffold structure (Table 7) and a maleimide core scaffold structure (Table 8) were identified. Thus, one embodiment of the invention additionally provides a core structure as disclosed herein and exemplified in Tables 7 and 8.

One embodiment of the invention further provides a core structure having the core structure I depicted below:

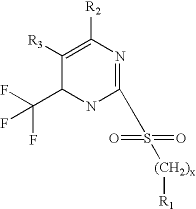

wherein R1 is independently selected from the group consisting of hydrogen; alkyloxoimino monocyclic ring wherein the alkyl is 1 to 10 carbon atoms and wherein each carbon atom may independently be substituted and wherein the monocyclic ring system comprises 5 to 7 ring carbon atoms and wherein 1 to 3 of the ring carbon atoms may independently be replaced by a hetero atom wherein the hetero atom is independently selected from nitrogen, oxygen and sulfur, and wherein the monocyclic ring may be fully saturated, partially saturated or unsaturated, and wherein any ring atom may independently be substituted;

alkyloxomonocyclic ring wherein the alkyl is 1 to 10 carbon atoms and wherein each carbon atom may independently be substituted and wherein the monocyclic ring system comprises 5 to 7 ring carbon atoms and wherein 1 to 3 of the ring carbon atoms may independently be replaced by a hetero atom wherein the hetero atom is independently selected from nitrogen, oxygen and sulfur, and wherein the monocyclic ring may be fully saturated, partially saturated or unsaturated, and wherein any ring atom may independently be substituted;

alkyloxooxyalkyl wherein the alkyl is 1 to 10 carbon and wherein each carbon atom may independently be substituted;

alkyloxoinimoalkyl wherein the alkyl is 1 to 10 carbon atoms and wherein each carbon atom may independently be substituted;

alkyloxoinimoalkyloxyalkyl wherein the alkyl is 1 to 10 carbon atoms and wherein each carbon atom may independently be substituted;

R2 is independently selected from the group consisting of hydrogen; monocyclic ring wherein the monocyclic ring system comprises 5 to 7 ring carbon atoms and wherein 1 to 3 of the ring carbon atoms may independently be replaced by a hetero atom wherein the hetero atom is independently selected from nitrogen, oxygen and sulfur, and wherein the monocyclic ring may be fully saturated, partially saturated or unsaturated, and wherein any ring atom may independently be substituted;

R3 is independently selected from hydrogen; monocyclic ring having 4 to 6 ring carbon atoms wherein said monocyclic ring may be fully saturated, partially saturated or unsaturated and wherein any ring atom may independently be substituted; and wherein x is an integer from 1 to 3.

One embodiment of the invention additionally provides a core structure having the core structure II depicted below:

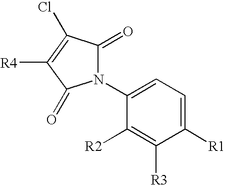

wherein R1, R2 and R3 are each independently selected from the group consisting of hydrogen; halogen, wherein the halogen is independently selected from the group consisting of fluorine, chlorine, bromine and iodine; and oxy; and

R4 is selected from the group consisting of iminoheterocyclic ring having 5 to 7 ring atoms wherein at least one of said ring atoms is replaced by a hetero atom wherein said hetero atom is independently selected from oxygen, nitrogen and sulfur, and wherein said ring may be fully saturated, partially saturated or unsaturated; and said iminoheterocyclic ring may be independently substituted one or more times by an alkyl group having 1 to 10 carbon atoms, an oxy group, and a monocyclic ring having 5 to 7 ring carbon atoms wherein said monocyclic ring may be fully saturated, partially saturated or unsaturated.

One embodiment of the invention further provides a core structure having the core structure III depicted below:

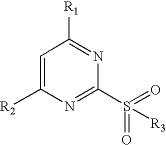

Exemplary R groups for core structure III are described in Table 7 below. Examples of R groups include, but not limited to, R1: hydrogen, ethyl formate, ethyl acetate, N-(5-methylisoxazol-3-yl)propionamide, 1-morpholinopropan-1-one, 1-(2-methylpiperidin-1-yl)propan-1-one, N-isopropylpropionamide, N-(1-methoxypropan-2-yl)acetamide, N-(5-isopropyl-1,3,4-thiadiazol-2-yl)acetamide, N-(5-methylisoxazol-3-yl)acetamide, N-(5-methylisoxazol-3-yl)propionamide; R2: thiophene, phenyl, anisole, 3-fluorobenzene, cyclohexane, 3-methylcyclohexane, furan, 3,4-dimethoxybenzene; and R3: hydrogen, cyclohexane, 3-methylcyclohexane.

One embodiment of the invention also provides a core structure having the core IV structure depicted below:

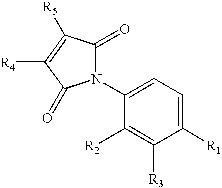

Exemplary R groups for core structure IV are described in Table 8 below. Examples of R groups include, but not limited to, R1, R2 and R3: hydrogen, methoxy, chloride; and R4: hydrogen, morpholine, 3-methylpiperazine, 1,5-dimethyl-4-(methylamino)-2-phenyl-1H-pyrazol-3(2H)-one. The R5 group of core structure II can be selected from the halogens fluorine, chlorine, bromine, and iodine.

One embodiment of the invention additionally provides analogs of such compounds, including analogs and derivatives of the compounds shown in Tables 2-8, provided below, or having a core structure selected from core structures I-IV. As described in the examples and shown in Tables 3-5, 7 and 8 various analogs are provided, in which R groups are varied. It is understood that analogs of the compounds disclosed herein, including those shown in Tables 2-8 or in core structures I-IV, can be readily prepared by one skilled in the art using well known methods of chemical synthesis and performing structure activity relationship (SAR) studies (see Example 5 below).

It is understood that various modifications can be made to the compounds shown in Tables 2-8 or in the core structures disclosed herein to generate analogs using well known methods. Examples of such analogs are provided in Tables 3-5 below. It is further understood that the R groups in the various core structures can be varied. Furthermore, one skilled in the art can readily determine the activity of various analogs using the methods disclosed herein.

In some embodiments, the compound can be a compound selected from the compounds listed in Tables 2-8 below. In some embodiments, the compound can be MLS-0067130.

The compounds of various embodiments disclosed herein, or analogs thereof, can also be described or characterized according to other moieties or combinations of moieties that, when present, renders the agent capable of binding to an anti-apoptotic Bcl-2 polypeptide. Such moieties can be, for example, the R groups of core structures as disclosed herein and, thus, the R groups can be selected from the moieties shown in Tables 7 and 8 as well as those described below. Definitions for various moieties that can be present in the compounds of various embodiments of the invention and analogs thereof are set forth below.

### DEFINITIONS AND GENERAL PARAMETERS

The following definitions are set forth to illustrate and define the meaning and scope of the various terms used to describe various embodiments of the invention herein.

As used herein, the term “alkyl,” alone or in combination, refers to a saturated, straight-chain or branched-chain hydrocarbon moiety containing from 1 to 10, in particular from 1 to 6, for example, from 1 to 4, carbon atoms. Examples of such moieties include, but are not limited to, methyl, ethyl, n-propyl, iso-propyl, n-butyl, iso-butyl, sec-butyl, tert-butyl, pentyl, iso-amyl, hexyl, decyl and the like.

The term “alkene,” alone or in combination, refers to a straight-chain or branched-chain hydrocarbon moiety having at least one carbon-carbon double bond in a total of from 2 to 10, in particular from 2 to 6, for example, from 2 to 4, carbon atoms. Examples of such moieties include, but are not limited to, ethenyl, E- and Z-propenyl, isopropenyl, E- and Z-butenyl, E- and Z-isobutenyl, E- and Z-pentenyl, decenyl, methylidene (═CH2), ethylidene (—CH═CH—), propylidene (—CH2-CH═CH—) and the like.

The term “alkyne,” alone or in combination, refers to a straight-chain or branched-chain hydrocarbon moiety having at least one carbon-carbon triple bond in a total of from 2 to 10, in particular from 2 to 6, for example, from 2 to 4, carbon atoms. Examples of such moieties include, but are not limited to, ethynyl (acetylenyl), propynyl (propargyl), butynyl, hexynyl, decynyl and the like.

The term “cycloalkyl,” alone or in combination, refers to a saturated, cyclic arrangement of carbon atoms which number from 3 to 8, for example, from 3 to 6, carbon atoms. Examples of such cycloalkyl moieties include, but are not limited to, cyclopropyl, cyclobutyl, cyclopentyl, cyclohexyl and the like.

The term “aryl” refers to a carbocyclic (consisting entirely of carbon and hydrogen) aromatic group selected from the group consisting of phenyl, naphthyl, indenyl, indanyl, azulenyl, fluorenyl, and anthracenyl; or a heterocyclic aromatic group selected from the group consisting of furyl, thienyl, pyridyl, pyrrolyl, oxazolyl), thiazolyl, imidazolyl, pyrazolyl, 2-pyrazolinyl, pyrazolidinyl, isoxazolyl, isothiazolyl, 1,2,3-oxadiazolyl, 1,2,3-tri azolyl, 1,3,4-thiadiazolyl, pyridazinyl, pyrimidinyl. pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, and phenoxazinyl.

“Aryl” groups, as defined in this application may independently contain one to four substituents which are independently selected from the group consisting of hydrogen, halogen, hydroxyl, amino, nitro, trifluoromethyl, trifluoromethoxy, alkyl, alkenyl, alkynyl, cyano, carboxy, carboalkoxy, 1,2-dioxyethylene, alkoxy, alkenoxy or alkynoxy, alkylamino, alkenylamino, alkynylamino, aliphatic or aromatic acyl, alkoxy-carbonylamino, alkylsulfonylamino, morpholinocarbonylamino, thiomorpholinocarbonylamino, N-alkyl guanidino, aralkylaminosulfonyl; aralkoxyalkyl; N-aralkoxyurea; N-hydroxylurea; N-alkenylurea; N,N-(alkyl, hydroxyl)urea; heterocyclyl; thioaryloxy-substituted aryl; N,N-(aryl, alkyl)hydrazino; Ar′-substituted sulfonylheterocyclyl; aralkyl-substituted heterocyclyl; cycloalkyl and cycloakenyl-substituted heterocyclyl; cycloalkyl-fused aryl; aryloxy-substituted alkyl; heterocyclylamino; aliphatic or aromatic acylaminocarbonyl; aliphatic or aromatic acyl-substituted alkenyl; Ar′-substituted aminocarbonyloxy; Ar′,Ar′-disubstituted aryl; aliphatic or aromatic acyl-substituted acyl; cycloalkylcarbonylalkyl; cycloalkyl-substituted amino; aryloxycarbonylalkyl; phosphorodiamidyl acid or ester;

“Ar′” is a carbocyclic or heterocyclic aryl group as defined above having one to three substituents selected from the group consisting of hydrogen, halogen, hydroxyl, amino, nitro, trifluoromethyl, trifluoromethoxy, alkyl, alkenyl, alkynyl, 1,2-dioxymethylene, 1,2-dioxyethylene, alkoxy, alkenoxy, alkynoxy, alkylamino, alkenylamino or alkynylamino, alkylcarbonyloxy, aliphatic or aromatic acyl, alkylcarbonylamino, alkoxycarbonylamino, alkylsulfonylamino, N-alkyl or N,N-dialkyl urea.

The term “alkoxy,” alone or in combination, refers to an alkyl ether moiety, wherein the term “alkyl” is as defined above. Examples of suitable alkyl ether moieties include, but are not limited to, methoxy, ethoxy, n-propoxy, iso-propoxy, n-butoxy, iso-butoxy, sec-butoxy, tert-butoxy and the like.

The term “alkenoxy,” alone or in combination, refers to a moiety of formula alkenyl-O—, wherein the term “alkenyl” is as defined above. Examples of suitable alkenoxy moieties include, but are not limited to, allyloxy, E- and Z-3-methyl-2-propenoxy and the like.

The term “thioalkoxy” refers to a thioether moiety of formula alkyl-S—, wherein alkyl is as defined above.

The term “alkylamino,” alone or in combination, refers to a mono- or di-alkyl-substituted amino group (i.e., a group of formula alkyl-NH— or (alkyl)2-N—), wherein the term “alkyl” is as defined above. Examples of suitable alkylamino moieties include, but are not limited to, methylamino, ethylamino, propylamino, isopropylamino, t-butylamino, N,N-diethylamino and the like.

The term “amide” refers to either —N(R1)-C(═O)— or —C(═O)—N(R1)- where (R1) is defined herein to include hydrogen as well as other groups. The term “substituted amide” refers to the situation where (R1) is not hydrogen, while the term “unsubstituted amide” refers to the situation where (R1) is hydrogen.

The term “aryloxy,” alone or in combination, refers to a moiety of formula aryl-O—, wherein aryl is as defined above. Examples of aryloxy moieties include, but are not limited to, phenoxy, naphthoxy, pyridyloxy and the like.

The term “arylamino,” alone or in combination, refers to a moiety of formula aryl-NH—, wherein aryl is as defined above. Examples of arylamino moieties include, but are not limited to, phenylamino (anilido), naphthylamino, 2-, 3- and 4-pyridylamino and the like.

The term “aryl-fused cycloalkyl,” alone or in combination, refers to a cycloalkyl moiety which shares two adjacent atoms with an aryl moiety, wherein the terms “cycloalkyl” and “aryl” are as defined above. An example of an aryl-fused cycloalkyl moiety is a benzofused cyclobutyl group.

The term “alkylcarbonylamino,” alone or in combination, refers to a moiety of formula alkyl-CONH, wherein the term “alkyl” is as defined above.

The term “alkoxycarbonylamino,” alone or in combination, refers to a moiety of formula alkyl-OCONH—, wherein the term “alkyl” is as defined above.

The term “alkylsulfonylamino,” alone or in combination, refers to a moiety of formula alkyl-SO2NH—, wherein the term “alkyl” is as defined above.

The term “arylsulfonylamino,” alone or in combination, refers to a moiety of formula aryl-SO2NH—, wherein the term “aryl” is as defined above.

The term “N-alkylurea,” alone or in combination, refers to a moiety of formula alkyl-NH—CO—NH—, wherein the term “alkyl” is as defined above.

The term “N-arylurea,” alone or in combination, refers to a moiety of formula aryl-NH—CO—NH—, wherein the term “aryl” is as defined above.

The term “halogen” means fluorine, chlorine, bromine and iodine.

In view of the above definitions, other chemical terms used throughout this application can be easily understood by those of skill in the art. Terms may be used alone or combined to describe a combination of moieties according to accepted chemical nomenclature.

In addition, one embodiment of the invention provides a complex having an anti-apoptotic Bcl-2 polypeptide bound to a compound, for example, a compound as shown in Tables 2-8 or a core structure disclosed herein. The complex can be isolated from at least one other cellular component normally occurring with the anti-apoptotic Bcl-2 polypeptide in nature. For example, the complex can be in a purified state being substantially free of other cellular components that normally occur with the anti-apoptotic Bcl-2 polypeptide in nature. The complex can also occur in a genetically engineered cell that does not normally express the anti-apoptotic Bcl-2 polypeptide.

As used herein, an “anti-apoptotic Bcl-2 polypeptide” refers to a member of the Bcl-2 family that exhibits anti-apoptotic activity (Reed and Pellecchia, Blood 106:408-418 (2005)). Exemplary anti-apoptotic Bcl-2 polypeptides include Bcl-2, Bcl-XL, Mcl-1, Bcl-W, Bfl-1 and Bcl-B of humans and their orthologs and homologs from other species, such as Boo/Diva and A1 of mice and Ced9 of Caenorhabditis elegans. It is understood that a Bcl-2 polypeptide can include a variant of an anti-apoptotic Bcl-2 polypeptide so long as the variant retains anti-apoptotic activity, including variants and homologs from different species, including human, non-human primate, mouse, rat, hamster, or other mammalian species or non-mammalian species. Exemplary human anti-apoptotic Bcl-2 polypeptides include Bcl-2 (GenBank accession Nos. NM_{—}000633, Mar. 11, 2007; NM_{—}000657, Mar. 11, 2007); Bcl-XL (GenBank accession No. Z23115, Jul. 26, 1994); Mcl-1 (GenBank accession Nos. NM_{—}182763, Mar. 25, 2007; NM_{—}021960, Mar. 25, 2007); Bcl-W (GenBank accession No. U59747, Sep. 29, 1996); Bfl-1 (GenBank accession No. U27467, Nov. 29, 1995); and Bcl-B (GenBank accession No. AF326964, May 1, 2001), each of which is incorporated herein by reference. A variant can also include amino acid insertions, deletions and substitutions, so long as the Bcl-2 polypeptide variant retains anti-apoptotic activity. For example, deletion of the C-terminal hydrophobic transmembrane domain from anti-apoptotic Bcl-2 family proteins can be useful for establishing convenient assays, especially for solution based assays where solubility of assay components can be a consideration. One skilled in the art can readily determine the anti-apoptotic activity of such variants based on the anti-apoptotic activity of known anti-apoptotic Bcl-2 polypeptides or BH3 domain binding activity.

As used herein the term “inhibiting,” when used in reference to a polypeptide activity, is intended to mean a reduction in an activity associated with the polypeptide. For example, a polypeptide such as an anti-apoptotic Bcl-2 polypeptide exhibits anti-apoptotic activity, and measurably reducing the anti-apoptotic activity of an anti-apoptotic Bcl-2 polypeptide is considered “inhibiting” the activity. One skilled in the art can readily determine a reduction in anti-apoptotic activity of an anti-apoptotic Bcl-2 polypeptide. A wide variety of surrogate indicators of apoptosis or cell death can also be utilized since anti-apoptotic Bcl-2 family proteins suppress many molecular components of apoptotic and non-apoptotic cell death pathways. Therefore, monitoring the activity of one or more of these components can be used as a surrogate to assess the anti-apoptotic activity of an anti-apoptotic Bcl-2 polypeptide and the effect of compounds on such activity. In addition, as disclosed herein, anti-apoptotic Bcl-2 polypeptides have binding activity for Bcl-2 homology-3 (BH3) domains. Therefore, inhibiting an anti-Bcl-2 polypeptide activity such as binding to a BH3 domain is considered inhibiting an anti-apoptotic Bcl-2 activity (see Examples). It has also been shown that anti-apoptotic Bcl-2 family proteins suppress autophagy, a process that can lead to cell death (Pattingre and Levine, Cancer Res. 66:2885-2888 (2006)). Bcl-2 family proteins also regulate Ca²⁺ levels in the endoplasmic reticulum (ER) and a wide variety of molecular events associated with Ca²⁺ release from ER, as well as signaling events associated with ER stress and the accumulation of unfolded proteins in the ER (unfolded protein response (UPR))(Xu et al., J. Clin. Invest. 115:2656-2664 (2005)).

As used herein, the term “inhibitor” is interchangeably used to denote “antagonist”. Both these terms define compositions which have the capability of decreasing certain enzyme activity or competing with the activity or function of a substrate of said enzyme.

As described previously, small molecule inhibitors directly binding Bcl-2 or related anti-apoptotic proteins have entered clinical trials for cancer (Reed and Pellecchia, Blood 106:408-418 (2005)). For example, the natural product gossypol is in clinical trials and binds a hydrophobic pocket found on the surface of anti-apoptotic Bcl-2 family polypeptides. The binding pocket is a regulatory site where endogenous antagonists dock onto Bcl-2 and related anti-apoptotic proteins, mimicking their cytodestructive activity. Endogenous antagonists bind via a conserved 16 amino acid motif called a Bcl-2 homology-3 (BH3) domain. Thus, inhibiting the binding of an anti-apoptotic Bcl-2 polypeptide to a regulatory BH3 domain containing polypeptide is an indicator of a substance that mimics the BH3 domain and thus promotes apoptosis (Reed and Pellecchia, Blood 106:408-418 (2005)).

As used herein the term “isolated,” when used in reference to a compound, means that the compound is separated from one or more components of a natural source if the compound is a natural product or from reagent, precursor, or reaction product if the compound is a synthetic product produced by chemical synthesis. Therefore, an isolated compound is a compound that is free from one or more components found in the synthetic reaction or reaction pathway that produces the compound. Also included in the term is a compound that is free from one or more components that it is found in nature. An isolated compound also includes a substantially pure compound. The term can include a molecule that has been produced by a combinatorial chemistry method and separated from precursors and other products by chemical purification or by binding to a second molecule with sufficient stability to be co-purified with the second molecule. The term can include naturally occurring compounds such as products of biosynthetic reactions or non-naturally occurring compounds.

As used herein, “pharmaceutically or therapeutically acceptable carrier” refers to a carrier medium which does not interfere with the effectiveness of the biological activity of the active ingredients and which is minimally toxic to the host or patient.

As used herein, “stereoisomer” refers to a chemical compound having the same molecular weight, chemical composition, and constitution as another, but with the atoms grouped differently. That is, certain identical chemical moieties are at different orientations in space and, therefore, when pure, have the ability to rotate the plane of polarized light. However, some pure stereoisomers can have an optical rotation that is so slight that it is undetectable with present instrumentation. The compounds described herein can have one or more asymmetrical carbon atoms and therefore include various stereoisomers. All stereoisomers are included within the scope of the present invention.

As used herein, “therapeutically- or pharmaceutically-effective amount” as applied to the disclosed compositions refers to the amount of composition sufficient to induce a desired biological result. That result can be alleviation of the signs, symptoms, or causes of a disease, or any other desired alteration of a biological system. For example, the result can involve a decrease and/or reversal of cancerous cell growth.

As used herein, “homology” or “identity” or “similarity” refers to sequence similarity between two peptides or between two nucleic acid molecules. Homology can be determined by comparing a position in each sequence which can be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. An “unrelated” or “non-homologous” sequence shares less than about 40% identity, though preferably less than about 25% identity, with one of the sequences described herein.

As used herein “peptide” indicates a sequence of amino acids linked by peptide bonds.

Such modifications include, e.g., alkylation and, more specifically, methylation of one or more residues, insertion of or replacement of natural amino acid by non-natural amino acids, and replacement of an amide bond with other covalent bond. A peptidomimetic can optionally comprise at least one bond which is an amide-replacement bond such as urea bond, carbamate bond, sulfonamide bond, hydrazine bond, or any other covalent bond. The design of appropriate “peptidomimetic” can be computer assisted.

The term “spacer” denotes a chemical moiety whose purpose is to link, covalently, a cell-permeability moiety and a peptide or peptidomimetic. The spacer can be used to allow distance between the cell-permeability moiety and the peptide, or it is a chemical bond of any type. Linker denotes a direct chemical bond or a spacer.

The term “permeability” refers to the ability of an agent or substance to penetrate, pervade, or diffuse through a barrier, membrane, or a skin layer. “Cell permeability” or a “cell-penetration” moiety refers to any molecule known in the art which is able to facilitate or enhance penetration of molecules through membranes. Non-limiting examples include: hydrophobic moieties such as lipids, fatty acids, steroids and bulky aromatic or aliphatic compounds; moieties which can have cell-membrane receptors or carriers, such as steroids, vitamins and sugars, natural and non-natural amino acids and transporter peptides. Examples for lipid moieties which can be used are: Lipofectamine; Transfectace; Transfectam; Cytofectin; DMRIE; DLRIE; GAP-DLRIE; DOTAP; DOPE; DMEAP; DODMP; DOPC; DDAB; DOSPA; EDLPC; EDMPC; DPH; TMADPH; CTAB; lysyl-PE; DC-Cho; -alanyl cholesterol; DCGS; DPPES; DCPE; DMAP; DMPE; DOGS; DOHME; DPEPC; Pluronic; Tween; BRIJ; plasmalogen; phosphatidylethanolamine; phosphatidylcholine; glycerol-3-ethylphosphatidylcholine; dimethyl ammonium propane; trimethyl ammonium propane; diethylammonium propane; triethylammonium propane; dimethyldioctadecylammonium bromide; a sphingolipid; sphingomyelin; a lysolipid; a glycolipid; a sulfatide; a glycosphingolipid; cholesterol; cholesterol ester; cholesterol salt; oil; N-succinyldioleoylphosphatidylethanolamine; 1,2-dioleoyl-sn-glycerol; 1,3-dipalmitoyl-2 succinylglycerol; 1,2-dipalmitoyl-sn-3-succinylglycerol; 1-hexadecyl-2-palmitoylglycerophosphatidylethanolamine; palmitoylhomocysteine; N,N′-Bis(do-decyaminocarbonylmethylene)-N,N′-bis((-N,N,N-trimethylammoniumethyl-aminocarbonyl-methylene)ethylenediamine tetraiodide; N,N″ Bis(hexadecylaminocarbonylmethylene)-N,N′,N″-tris((-N,N,N-trimethylammonium-ethylaminocarbonylmethylenediethylenetri-aminehexaiodide; N,N′-Bis(dodecylaminocarbonylmethylene)-N,N″-bis((-N,N,N-trimethylammoniumethylamino-carbonylmethylene)cy-clohexylene-1,4-diaminetetra-iodide; 1,7,7-tetra-((N,N,N,N-tetramethylammoniumethylamino-carbonylmethylene)-3-hexadecylaminocarbonyl methylene-1,3,7-triaazaheptane heptaiodide; N,N,N′,N′-tetra((-N,N,N-trimethylammonium-ethylaminocarbonylmethylene)-N′-(1,2-dioleoylglycero-3-phosphoethanolaminocarbonyl methylene)diethylenetriamine tetraiodide; dioleoylphosphatidyl ethanolamine; a fatty acid; a lysolipid; phosphatidylcholine; phosphatidylethanolamine; phosphatidylserine; phosphatidylglycerol; phosphatidylinositol; a sphingolipid; a glycolipid; a glucolipid; a sulfatide; a glycosphingolipid; phosphatidic acid; palmitic acid; stearic acid; arachidonic acid; oleic acid; a lipid bearing a polymer; a lipid bearing a sulfonated saccharide; cholesterol; tocopherol hemisuccinate; a lipid with an ether-linked fatty acid; a lipid with an ester-linked fatty acid; a polymerized lipid; diacetyl phosphate; stearylamine; cardiolipin; a phospholipid with a fatty acid of 6-8 carbons in length; a phospholipid with asymmetric acyl chains; 6-(5cholesten-3b-yloxy)-1-thio-b-D-galactopyranoside; digalactosyldiglyceride; 6-(5-cholesten-3b-yloxy)hexyl-6-amino-6-deoxy-1-thio-b-D-galactopyranoside; 6-(5-cholesten-3b-yloxy)hexyl-6-amino-6-deoxyl-1-thio-a-D-mannopyranoside; 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl];-2-aminopalmitic acid; cholesteryl(4′-trimethyl-ammonio)butanoate; N-succinyldioleoyl-phosphatidylethanolamine; 1,2-dioleoyl-sn-glycerol; 1,2-dipalmitoyl-sn-3-succinyl-glycerol; 1,3-dipalmitoyl-2-succinylglycerol; 1-hexadecyl-2-palmitoylglycero-phosphoethanolamine; palmitoylhomocysteine; cyclic 9-amino-acid peptide as described in. Laakkonen et al. 2002 Nature Med 8:751-755; a peptide described in Porkka et al. 2002 PNAS USA 99:7444-7449; and polymers of L- or D-arginine as described in Mitchell et al. 2000 J Peptide Res 56:318-325.

As used herein, “cancer” and “cancerous” refer to any malignant proliferation of cells in a mammal.

“Inflammation” as used herein is a general term for the local accumulation of fluid, plasma proteins, and white blood cells that is initiated by physical injury, infection, or a local immune response. Many different forms of inflammation are associated with different diseases. “Inflammation-associated” diseases include, for example, lupus, psoriasis, rheumatoid arthritis, and inflammatory bowel disease. Other inflammation-associated diseases are discussed herein.

As used herein, “neurodegenerative disease” is a condition which affects brain function and is a result of deterioration of neurons. The neurodegenerative diseases are divided into two groups: a) conditions causing problems with movements, and conditions affecting memory and conditions related to dementia. Neurodegenerative diseases include, for example, Huntington's disease, spinocerebellar ataxias, Machado-Joseph disease, Spinal and Bulbar muscular atrophy (SBMA or Kennedy's disease), Dentatorubral Pallidoluysian Atrophy (DRPLA), Fragile X syndrome, Fragile XE mental retardation, Friedreich ataxia, myotonic dystrophy, Spinocerebellar ataxias (types 8, 10 and 12), spinal muscular atrophy (Werdnig-Hoffman disease, Kugelberg-Welander disease), Alzheimer's disease, amyotrophic lateral sclerosis, Parkinson's disease, Pick's disease, and spongiform encephalopathies. Additional neurodegenerative diseases include, for example, age-related memory impairment, agyrophilic grain dementia, Parkinsonism-dementia complex of Guam, auto-immune conditions (e.g., Guillain-Barre syndrome, Lupus), Biswanger's disease, brain and spinal tumors (including neurofibromatosis), cerebral amyloid angiopathies, cerebral palsy, chronic fatigue syndrome, corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, Dementia Pugilistica, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoff's syndrome, mild cognitive impairment, monomeric amyotrophy, motor neuron diseases, multiple system atrophy, multiple sclerosis and other demyelinating conditions (e.g., leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or viral infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyugeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, Wilson disease, Alexander disease, Alper's disease, ataxia telangiectasia, Canavan disease, Cockayne syndrome, Krabbe disease, multiple system atrophy, Pelizaeus-Merzbacher Disease, primary lateral sclerosis, Refsum's disease, Sandhoff disease, Schilder's disease, Steele-Richardson-Olszewski disease, tabes dorsalis.

When two compounds are administered in combination or used in combination therapy according to some embodiments, the term “combination” in this context means that the drugs are given contemporaneously, either simultaneously or sequentially. This term is exchangeable with the term “coadministration” which in the context of this invention is defined to mean the administration of more than one therapeutic in the course of a coordinated treatment to achieve an improved clinical outcome. Such coadministration can also be coextensive, that is, occurring during overlapping periods of time.

### Exemplary Embodiments

In some embodiments, for inhibiting an anti-apoptotic Bcl-2 polypeptide activity such as binding to a BH3 domain, the anti-apoptotic Bcl-2 polypeptide is contacted with an amount of compound effective to inhibit an anti-apoptotic Bcl-2 polypeptide activity such as binding to a BH3 domain. For example, in a method of promoting or increasing apoptosis in a cell, an effective amount of the compound is an amount that is sufficient to yield an increase in apoptosis in a cell compared to the absence of the compound. An increase in apoptosis can be determined using any of the well known methods for determining apoptotic activity, as disclosed herein.

In some embodiments, compounds can be contacted with a compound under conditions suitable to inhibit an anti-apoptotic Bcl-2 polypeptide activity such as binding to a BH3 domain. As used herein, it is understood that inhibiting binding to a BH3 domain includes inhibiting binding to an isolated BH3 domain as well as a BH3 domain contained within a larger polypeptide, such as an intact polypeptide or fragment thereof containing a BH3 domain. The compound that is contacted with the anti-apoptotic Bcl-2 polypeptide can be present in a mixture of compounds, in an isolated form or in substantially pure form. As described herein, a mixture of compounds can be contacted with an anti-apoptotic polypeptide in a screening method employing positional scanning or iteration. Such a mixture can be identified as having the ability to bind to an anti-apoptotic Bcl-2 polypeptide. The mixture can be used in the methods disclosed herein to inhibit an anti-apoptotic Bcl-2 polypeptide activity such as binding to a BH3 domain. Alternatively, a particular species in the mixture having such activity can be further defined by isolating individual species in the mixture and repeating the binding assay or performing a second assay for inhibitors of an anti-apoptotic Bcl-2 polypeptide activity such as binding to a BH3. A compound that binds to an anti-apoptotic Bcl-2 polypeptide can be contacted with the anti-apoptotic Bcl-2 polypeptide in a substantially pure form, as a conjugate or in a formulation, as described herein.

In some embodiments, methods of inhibiting an anti-apoptotic Bcl-2 polypeptide activity are provided. In some embodiments, methods are provided for inhibiting binding of an anti-apoptotic Bcl-2 polypeptide to a BH3 domain by contacting a Bcl-2 polypeptide with a compound that inhibits binding of a Bcl-2 polypeptide to a BH3 domain such as the compounds shown in Tables 2-8 or a core structure disclosed herein, thereby inhibiting binding of the Bcl-2 polypeptide to a BH3 domain. In various embodiments, an anti-apoptotic Bcl-2 polypeptide can be contacted with a compound disclosed herein in a cell. Accordingly, one embodiment of the invention additionally provides methods of promoting or increasing apoptosis in a cell, by contacting the cell with an effective amount of a compound that binds to an anti-apoptotic Bcl-2 polypeptide to inhibit binding of the Bcl-2 polypeptide to a BH3 domain, thereby inhibiting the anti-apoptotic activity of the Bcl-2 polypeptide. Thus, one embodiment of the invention provides methods of increasing apoptosis in a cell by contacting a cell with an effective amount of a compound, for example, a compound selected from the compounds shown in Tables 2-8 or from a core structure disclosed herein, whereby binding of an anti-apoptotic Bcl-2 polypeptide to a BH3 domain is inhibited and apoptosis is increased.

Methods described herein for cytosolic delivery of a compound, such as attachment of a moiety of a conjugate, can be used in a method of promoting or increasing apoptosis in a cell. An effective amount of the compound can be identified as an amount sufficient to allow apoptosis to occur in the cell. Methods of determining morphological changes in a cell or nucleus that are characteristic of apoptosis can be used to monitor apoptosis while performing a method of promoting or increasing apoptosis in a cell. Apoptosis in a cell can be determined by identifying morphological changes in a cell or a cell nucleus characteristic of apoptosis. Such changes that are characteristic of apoptosis include, for example, chromatin condensation, nuclear fragmentation, cell shrinkage, or cell blebbing leading to the eventual breakage into small membrane surrounded fragments termed apoptotic bodies. Thus, a compound that promotes or increases apoptosis can be identified according to the ability to cause a characteristic apoptotic change when added to a cell that contains an anti-apoptotic Bcl-2 polypeptide. A similar assay can be performed on a cell free extract derived from such a cell so long as an apoptotic change such as chromatin condensation or nuclear fragmentation can be distinguished in the presence and absence of the added compound.

Many other molecular events associated with apoptosis can also be employed as end points for monitoring the activity of Bcl-2 family proteins and antagonists or agonists of these proteins. These alternative end points include, but are not limited to, caspase protease activity, cleavage of caspases or their cellular substrates, release of proteins from mitochondria such as cytochrome c, apoptosis inducing factor (AIF), second mitochondrial activator of caspases (SMAC), endonuclease G (EndoG) and others, reductions in mitochondrial electrochemical gradient, and cell surface exposure of Annexin V-binding phospholipids. Also, because anti-apoptotic Bcl-2 family proteins also inhibit non-apoptotic cell death, other manifestations of cell death can provide suitable end points, including reduction in ATP cellular levels and accumulation of reactive oxygen species (ROS).

In some embodiments, the methods disclosed herein can be carried out in a cell from any organism in which apoptosis or non-apoptotic cell death can be modulated by an anti-apoptotic Bcl-2 polypeptide, for example, a eukaryotic cell, such as a mammalian cell, human cell, non human-primate cell, mouse cell, hamster cell, or other animal cell; an invertebrate cell such as a fly or nematode cell or a yeast cell. Various cell types can be used in the methods disclosed herein including, for example, a tumor cell, stem cell, neural cell, fat cell, hematopoietic cell, lymphoid cell, liver cell or muscle cell. In the case of Bfl-1, a particularly useful cell is a lymphoid cell or a macrophage, because such cells endogenously express high levels of Bfl-1. The methods disclosed herein are useful for inducing apoptosis in aberrantly regulated cells including, for example, cells that exhibit uncontrolled cell proliferation as well as cells that exhibit dysfunction in specific phases of the cell cycle, leading to altered proliferative characteristics or morphological phenotypes. Specific examples of aberrantly regulated cell types include neoplastic cells such as cancer and hyperplastic cells characteristic of tissue hyperplasia. Another specific example includes immune cells that become aberrantly activated or fail to down regulate following stimulation. Autoimmune diseases are mediated by such aberrantly regulated immune cells. Aberrantly regulated cells also include cells that are biochemically or physiologically dysfunctional. Other types of aberrant regulation of cell function or proliferation are known to those skilled in the art and are similarly characteristic of target cells applicable for apoptotic destruction using the methods disclosed herein

### Conjugates

Some embodiments disclosed herein provide conjugates including a moiety linked to a compound that inhibits an anti-apoptotic Bcl-2 polypeptide activity, such as, for example, inhibiting an anti-apoptotic Bcl-2 polypeptide from binding to a BH3 domain. Generally, a conjugate is generated by covalently cross-linking a moiety to a compound or by synthesizing the conjugate such that a covalent bond is formed between the compound and the moiety. A conjugate of the invention can include a moiety useful for targeting the compound to a particular cell or for increasing the stability or biological half life of the compound that inhibits an anti-apoptotic Bcl-2 polypeptide activity. For example, a moiety can be a particular antibody, functional fragment thereof, or other binding polypeptide that has specificity for a particular cell in which it is desired to promote or increase apoptosis, such as a tumor cell. Any moiety capable of targeting the compound to a cell in which an anti-apoptotic Bcl-2 polypeptide activity is to be inhibited can be used as a conjugate.

In some embodiments, a conjugate is provided comprising a compound of the invention selected from, for example, a compound shown in Tables 2-8 or from a core structure disclosed herein, conjugated to a detectable moiety. The detectable moiety can be, for example, a fluorophore.

A conjugate of a compound of the invention that inhibits an anti-apoptotic Bcl-2 polypeptide activity can also be a moiety capable of introducing the compound to the cytosol of a cell or otherwise facilitating passage of the compound through the cell membrane. A compound can be introduced into the cell by, for example, a heterologous targeting domain or using a lipid based carrier. Thus, one embodiment of the invention provides cytosolic delivery of a compound that inhibits an anti-apoptotic Bcl-2 polypeptide activity such as binding to a BH3 domain.

A moiety can also be a drug delivery vehicle such as a microdevice containing chambers, including nanoparticle devices, a cell, a liposome or a virus that provides stability or properties otherwise advantageous for administration of the compound that inhibits an anti-apoptotic Bcl-2 polypeptide activity. Generally, such microdevices, should be nontoxic and, if desired, biodegradable. Various moieties, including microcapsules, which can contain a compound, and methods for linking a moiety, including a chambered microdevice, to a therapeutic agent are well known in the art and commercially available (see, for example, Remington's Pharmaceutical Sciences 18th ed. (Mack Publishing Co. 1990), chapters 89-91; Harlow and Lane, Antibodies: A laboratory manual (Cold Spring Harbor Laboratory Press 1988); see, also, Hermanson, Bioconjugate Techniques, Academic Press, San Diego (1996).

In addition, a compound of the invention that inhibits an anti-apoptotic Bcl-2 activity can be included in a formulation that is incorporated into biodegradable polymers allowing for sustained release of the compound, the polymers being implanted in the vicinity of where drug delivery is desired, for example, at the site of a tumor or implanted so that the compound is released systemically over time. Osmotic minipumps also can be used to provide controlled delivery of specific concentrations of the compound and formulations through cannulae to the site of interest, such as directly into a tumor growth or into the vascular supply of a tumor. The biodegradable polymers and their use are described, for example, in detail in Brem et al., J. Neurosurg. 74:441-446 (1991).

A conjugate can include a moiety that is a label. For example, a compound of the invention can be labeled with a detectable moiety to generate a conjugate comprising a labeled compound. A labeled compound that binds to an anti-apoptotic Bcl-2 polypeptide can be used to identify the subcellular localization of the anti-apoptotic Bcl-2 polypeptide or to identify a previously unidentified anti-apoptotic Bcl-2 polypeptide. A labeled compound that binds to an anti-apoptotic Bcl-2 polypeptide can also be used to identify other molecules that interact with an anti-apoptotic Bcl-2 polypeptide. For example, a binding competition assay utilizing a labeled compound can be used to identify another compound that inhibits an anti-apoptotic Bcl-2 polypeptide activity such as binding to a BH3 domain by competition with the labeled compound. A label that can be incorporated as a moiety includes, for example, a fluorophore, chromophore, paramagnetic spin label, radionucleotide, or binding group having specificity for another molecule that can be detected, for example, biotin or haptens which can bind to a hapten-specific antibody, fluorescent proteins such as green fluorescent protein (GFP), or other proteins or peptides which can specifically bind to another molecule that can be detected, such as a labeled antibody.

A particularly useful detectable moiety is a fluorophore. Exemplary fluorophores are well known to those skilled in the art (see Hermanson, Bioconjugate Techniques, pp. 297-364, Academic Press, San Diego (1996); Molecular Probes, Eugene Oreg.). Rhodamine derivatives include, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like. Other suitable fluors include 7-nitrobenz-2-oxa-1,3-diazole (NBD).

Additional exemplary fluorophores include, for example, fluorescein and derivatives thereof. Other fluorophores include napthalenes such as dansyl (5-dimethylaminonapthalene-1-sulfonyl). Additional fluorophores include coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like.

Other fluorophores include 4,4-difluoro-4-bora-3a,4a-diaza-s-indacene (BODIPY™) and derivatives thereof (Molecular Probes; Eugene Oreg.). Further fluorophores include pyrenes and sulfonated pyrenes such as Cascade Blue™ and derivatives thereof, including 8-methoxypyrene-1,3,6-trisulfonic acid, and the like. Additional fluorophores include pyridyloxazole derivatives and dapoxyl derivatives (Molecular Probes). Additional fluorophores include Lucifer Yellow (3,6-disulfonate-4-amino-naphthalimide) and derivatives thereof. CyDye™ fluorescent dyes (Amersham Pharmacia Biotech; Piscataway N.J.) can also be used.

In some embodiments, a labeled compound of the invention can be useful for identifying cells within a tissue that are inhibited from apoptosis by an anti-apoptotic Bcl-2 polypeptide. Thus, the labeled compound can be used in a diagnostic method to identify cells for which administration of a compound that inhibits an anti-apoptotic Bcl-2 polypeptide activity will allow apoptosis to proceed. The method can include the steps of administering a labeled compound to a tissue and identifying one or more cells that incorporate the labeled compound. The labeled compound can be administered using methods for in vivo delivery as described herein. The diagnostic methods can be used at a variety of resolutions. For example, the method can be carried out to identify a tissue containing cells labeled by the compound. Alternatively, higher resolution methods can be used to identify a particular cell or cell type within a tissue that is labeled in the presence of an anti-apoptotic Bcl-2 polypeptide. Because the diagnostic methods can be used to distinguish a cell for which administration of a compound will allow apoptosis to proceed from non-labeled cells, the methods can be useful for guiding the choice of targeting or delivery conjugate to use in a therapeutic method disclosed herein.

In some embodiments, the diagnostic method can be performed in vitro, in which case the labeled agent can be administered by injection or by soaking a tissue sample in a solution containing the labeled agent. Again the methods can be used at a resolution sufficient to distinguish within a tissue a cell having an anti-apoptotic Bcl-2 polypeptide over those that do not and therefore are not inhibited from apoptosis in this way. Such resolution can be achieved, for example, by use of a microscopic based technique. Further resolution can provide subcellular localization of an anti-apoptotic Bcl-2 polypeptide. Subcellular localization can be used to determine an appropriate cytosolic delivery conjugate or to further identify the role of apoptosis in the particular tissue or cells under study.

### Pharmaceutical Compositions

One embodiment of the invention also provides pharmaceutical compositions containing a compound and a pharmaceutical carrier. Such compositions can be used, for example, in the apoptosis promoting methods disclosed herein to inhibit, treat or reduce the severity of, or ameliorate a sign and/or symptom associated with, a pathological condition characterized by a pathologically reduced level of apoptosis. For example, a compound that inhibits an anti-apoptotic Bcl-2 polypeptide activity can be administered as a solution or suspension together with a pharmaceutically acceptable medium.

As used herein, the term “pharmaceutically acceptable carrier” is intended to mean a medium having sufficient purity and quality for use in humans. Such a medium can be a human pharmaceutical grade, sterile medium, such as water, sodium phosphate buffer, phosphate buffered saline, normal saline or Ringer's solution or other physiologically buffered saline, or other solvent or vehicle such as a glycol, glycerol, an oil such as olive oil or an injectable organic ester. Pharmaceutically acceptable media are substantially free from contaminating particles and organisms.

Formulations containing a compound of the invention include those applicable for parenteral administration such as subcutaneous, intraperitoneal, intramuscular, intravenous, intradermal, intracranial, intratracheal, and epidural administration. Additional formulations are applicable for oral, rectal, ophthalmic (including intravitreal or intracameral), nasal, topical (including buccal and sublingual), intrauterine, or vaginal administration. A formulation containing a compound can be presented in unit dosage form and can be prepared by pharmaceutical techniques well known to those skilled in the art. Such techniques include the step of bringing into association the active ingredient and a pharmaceutical carrier or excipient.

Formulations suitable for parenteral administration include aqueous and non-aqueous sterile injection solutions such as the pharmaceutically acceptable media described above. The solutions can additionally contain, for example, anti-oxidants, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

A pharmaceutically acceptable medium can additionally contain physiologically acceptable compounds that act, for example, to stabilize the compounds disclosed herein. Such physiologically acceptable compounds include, for example, carbohydrates such as glucose, sucrose or dextrans; antioxidants such as ascorbic acid or glutathione; chelating agents such as ethylenediaminetetraacetic acid (EDTA), which disrupts microbial membranes; divalent metal ions such as calcium or magnesium or other divalent cations; low molecular weight proteins; lipids or liposomes; or other stabilizers or excipients. As described previously, a formulation containing a compound also can be formulated with a pharmaceutically acceptable medium such as a biodegradable polymer. All of the above-described pharmaceutical carriers and media can be what is termed in the art pharmaceutical grade which means that they are of sufficient purity and quality for use in humans and are distinguishable from comparable reagents in research grade formulations.

One embodiment of the invention also provides a composition including an inhibitor of an anti-apoptotic Bcl-2 polypeptide activity and a molecule having therapeutic activity. A molecule included with a compound can be a molecule having activity against a condition characterized by a pathologically reduced level of apoptosis. For example, the compound can have activity against cancer or an autoimmune disease.

### Treatment of Pathological Conditions

One embodiment of the invention additionally provides a method of reducing the severity of a pathological condition in an individual by administering to an individual having a pathological condition characterized by a pathologically reduced level of apoptosis a compound selected from the compounds shown in Tables 2-8 or a core structure disclosed herein, or a derivative thereof, whereby binding of an anti-apoptotic Bcl-2 polypeptide to a BH3 domain is inhibited and the severity of the pathological condition is reduced. The pathological condition can be, for example, cancer or other conditions such as psoriasis, hyperplasia, an autoimmune disease, an inflammation-related disorder and restenosis. Examples of conditions characterized by pathologically reduced levels of apoptosis that can be treated in a method of one embodiment of the invention include, but are not limited to, restenosis; autoimmune disease such as lupus or rheumatoid arthritis; allograft rejection, proliferative lesions of the skin such as eczema; or benign prostate hypertrophy.

In some embodiments, methods for treating cancer by inducing apoptosis of cancer cells in an afflicted individual are provided. Accordingly, one or more inducers of apoptosis is administered to a patient in need of such treatment. A therapeutically effective amount of the drug can be administered as a composition in combination with a pharmaceutical vehicle. In other embodiments of the invention the apoptosis modulator targets a death antagonist associated with virally infected cells or self-reacting lymphocytes to comprise a treatment for viral infection or autoimmune disease.

For a review of apoptosis in the pathogenesis of disease, see, e.g., Thompson, 1995 Science 267:1456-1462. In particular, pro-apoptotic modulators of anti-apoptotic Bcl-2 polypeptides can be used to treat any condition characterized by the accumulation of cells which are regulated by anti-apoptotic Bcl-2 polypeptides. By “regulated by Bcl-2” with respect to the condition of a cell is meant that the balance between cell proliferation and apoptotic cell death is controlled, at least in part, by anti-apoptotic Bcl-2 polypeptides. For the most part, the cells express or overexpress anti-apoptotic Bcl-2 polypeptides. Enhancement of anti-apoptotic Bcl-2 polypeptides expression has been demonstrated to increase the resistance of cells to almost any apoptotic signal (Hockenbery et al. 1990 Nature 348:334; Nunez et al. 1990 Immunol 144:3602; Vaux et al. 1988 Nature 335:440; Hockenbery et al. 1993 Cell 75:241; Ohmori et al. 1993 Res Commun 192:30; Lotem et al. 1993 Cell Growth Differ 4:41; Miyashita et al. 1993 Blood 81:115). Principally, the proliferative disorders associated with the inhibition of cell apoptosis include cancer, autoimmune disorders and viral infections. Overexpression of anti-apoptotic Bcl-2 polypeptides specifically prevents cells from initiating apoptosis in response to a number of stimuli (Hockenbery et al. 1990 Nature 348:334; Nunez et al. 1990 J Immunol 144:3602; Vaux et al. 1988 Nature 335:440; Hockenbery et al. 1993 Cell 75:241). The induction of genes that inhibit anti-apoptotic Bcl-2 polypeptides can induce apoptosis in a wide variety of tumor types, suggesting that many tumors continually rely on Bcl-2 or related gene products to prevent cell death. Expression of anti-apoptotic Bcl-2 polypeptides has been associated with a poor prognosis in at least prostatic cancer, colon cancer and neuroblastoma (McDonnell et al. 1992 Cancer Res 52:6940; Hague et al. 1994 Oncogene 9:3367; Castle et al. 1993 Am J Pathol 143:1543). Bcl-2 or the related gene has been found to confer resistance to cell death in response to several chemotherapeutic agents (Ohmon et al. 1993 Res Commun 192:30; Lotem et al. 1993 Cell Growth Differ 4:41; Miyashita et al. 1993 Blood 81:115).

An effective amount of a compound that inhibits an anti-apoptotic Bcl-2 polypeptide activity such as binding to a BH3 domain, when used to treat a pathological condition, is an amount required to allow an increase in apoptosis in a target cell when administered to an individual. The dosage of a compound required to be therapeutically effective will depend, for example, on the pathological condition to be treated, the route and form of administration, the weight and condition of the individual, and previous or concurrent therapies. The appropriate amount considered to be an effective dose for a particular application of the method can be determined by those skilled in the art using the guidance provided herein and well known methods. For example, the amount can be extrapolated from in vitro or in vivo assays. One skilled in the art will recognize that the condition of the patient can be monitored throughout the course of therapy and that the amount of the compound that is administered can be adjusted accordingly.

For treating or reducing the severity of a pathological condition, an effective amount is an efficacious amount of the compound capable of increasing apoptosis that is pathologically reduced. An effective amount can be, for example, between about 10 μg/kg to 500 mg/kg body weight, for example, between about 0.1 mg/kg to 100 mg/kg, or preferably between about 1 mg/kg to 50 mg/kg, depending on the treatment regimen. For example, if a compound or formulation containing the compound is administered from one to several times a day, then a lower dose would be needed than if a formulation were administered weekly, or monthly or less frequently. Similarly, formulations that allow for timed-release of the compound, such as those described herein, would provide for the continuous release of a smaller amount of the compound than would be administered as a single bolus dose. For example, a compound of the invention can be administered at between about 1-5 mg/kg/week.

Formulations of compounds of the invention, variants and combinations thereof can also be delivered in alternating administrations so as to combine their apoptosis increasing effects over time. For example, a compound can be administered in a single bolus dose followed by multiple administrations of one or more formulations of the compound alone, or in combination with a different formulation of such a compound or formulation of a different compound, including one or more additional therapeutic agents, as discussed herein. Whether simultaneous or alternating delivery of the compound formulation, variations of the formulation and/or one or more additional therapeutic agents, the mode of administration can be any of those types of administrations described herein and will depend on the particular therapeutic need and efficacy of the compound selected for the purpose. Determining which compound, formulation, and variants thereof, including additional therapeutic agents, to combine in a temporally administered regime, will depend on the pathological condition to be treated and the specific physical characteristics of the individual affected with the disease. Those skilled in the art will know or can determine a specific regime of administration which is effective for a particular application using the teachings and guidance provided herein together with diagnostic and clinical criteria known within the field of art of the particular pathological condition.

The methods of treating a pathological condition characterized by pathologically reduced apoptosis additionally can be practiced in conjunction with other therapies, as disclosed herein. For example, for treating cancer, the methods disclosed herein can be practiced prior to, during, or subsequent to conventional cancer treatments such as surgery, chemotherapy, including administration of cytokines and growth factors, radiation or other methods known in the art.

Such treatments can act in a synergistic manner, with the reduction in tumor mass caused by the conventional therapy increasing the effectiveness of a compound, and vice versa. Non-limiting examples of therapeutic agents that are anti-cancer drugs that are suitable for co-administration with a compound are well known to those skilled in the art of cancer therapy. Anti-cancer drugs can be used in a composition with an inhibitor of an anti-apoptotic Bcl-2 polypeptide activity including, but not limited to, an alkylating agent such as mechlorethamine (nitrogen mustard), chlorambucil, cyclophosphamide, melphalan, ifosfamide; an antimetabolite such as methotrexate (MTX), 6-mercaptopurine, 5-fluorouracil (5-FU) or cytarabine; an antibody such as Rituxan™ (rituximab), Herceptin™ (trastuzumab), or Mabthera™ (rituximab); a plant alkaloid such as vinblastine or vincristine, or etoposide (VP16-213); an antibiotic such as doxorubicin, daunomycin, bleomycin, or mitomycin; a nitrosurea such as carmustine (BCNU) or lomustine (CCNU); an inorganic ion such as cisplatin (cis-DDP); a biological response modifier, for example, an interferon such as interferon alpha; an enzyme such as aspariginase; or a hormone such as tamoxifen or flutamide. These and other anti-cancer compounds, including those described herein below with respect to practicing a therapeutic method disclosed herein in combination with another therapeutic method, are known in the art and formulations suitable for pharmaceutical use are known as described, for example, in The Merck Manual 16th Ed., Merck Res. Labs., Rahway N.J. (1992). In addition, for treating a condition characterized by a pathologically reduced level of apoptosis, a compound can be administered in conjunction with a therapeutic antibody. Such a therapeutic antibody can be, for example, an antibody that modulates apoptosis, such as by binding to an apoptosis regulatory molecule and modulating its activity. As a non-limiting example, a compound can be administered in conjunction with an antibody that activates caspase 3, caspase 7, Trail-R1 or Trail R-2. Exemplary Trail-R1 and Trail-R2 monoclonal antibodies are available from, for example, Human Genome Sciences, Rockville, Md.

Additional anti-cancer agents include aminoglutethimide, amsacrine (m-AMSA), azacitidine, busulfan, carboplatin, dacarbazine, dactinomycin, daunorubicin, erythropoietin, estramustine, etoposide (VP16-213), floxuridine, hexamethylmelamine (HMM), hydroxyurea (hydroxycarbamide), interleukin 2, leuprolide acetate (LHRH-releasing factor analogue), mesna, mitoguazone (methyl-GAG, methyl glyoxal bis-guanylhydrazone, MGBG), mitotane, mitoxantrone, octreotide, pentostatin, plicamycin, procarbazine, semustine (methyl-CCNU), streptozocin, teniposide (VM-26), thioguanine, thiotepa, and vindesine. Furthermore, anti-cancer drugs including, for example, any of those set forth above with regard to combination compositions, can be administered prior to, during, or subsequent to administration of a compound in a method of treatment.

Treatment of pathological conditions of autoimmune disorders also can be accomplished by combining the compounds disclosed herein with conventional treatments for the particular autoimmune diseases. Conventional treatments include, for example, chemotherapy, steroid therapy, insulin and other growth factor and cytokine therapy, passive immunity, inhibitors of T cell receptor binding and T cell receptor vaccination, and the like.

The compounds disclosed herein can be administered in conjunction with these or other treatment methods known in the art and at various times prior, during or subsequent to initiation of conventional treatments. For a description of treatments for pathological conditions characterized by aberrant cell growth see, for example, The Merck Manual, Sixteenth Ed, (Berkow, R., Editor) Rahway, N.J., 1992, which is incorporated herein by reference.

As described above, administration of a formulation of a compound can be, for example, simultaneous with or delivered in alternative administrations with the conventional therapy, including multiple administrations. Simultaneous administration can be, for example, together in the same formulation or in different formulations delivered at about the same time or immediately in sequence. Alternating administrations can be, for example, delivering a compound and a conventional therapeutic treatment in temporally separate administrations. As described herein, the temporally separate administrations of a compound and conventional therapy can similarly use different modes of delivery and routes.

A condition characterized by a pathologically reduced level of apoptosis that can be treated using the compounds and methods disclosed herein include, for example, cancer, hyperplasia, autoimmune disease and restenosis. As used herein, the term “cancer” is intended to mean a class of diseases characterized by the uncontrolled growth of aberrant cells, including all known cancers, and neoplastic conditions, whether characterized as malignant, benign, soft tissue or solid tumor. Specific cancers include digestive and gastrointestinal cancers, such as anal cancer, bile duct cancer, gastrointestinal carcinoid tumor, colon cancer, esophageal cancer, gallbladder cancer, liver cancer, pancreatic cancer, rectal cancer, appendix cancer, small intestine cancer and stomach (gastric) cancer; breast cancer; ovarian cancer; lung cancer; renal cancer; central nervous system (CNS) cancer, including brain cancer; prostate cancer; hematopoietic neoplasms such as leukemia, lymphoma and melanoma; skin cancers, eye cancers, and the like.

A growing number of human diseases have been classified as autoimmune and include, for example, rheumatoid arthritis, myasthenia gravis, multiple sclerosis, psoriasis, systemic lupus erythematosus, autoimmune thyroiditis, Graves' disease, inflammatory bowel disease, autoimmune uveoretinitis, polymyositis and diabetes. Animal models for many conditions characterized by a pathologically reduced level of apoptosis have been developed and can be employed for predictive assessment of therapeutic treatments employing a compound. Moreover, pharmaceutical compositions of a compound can be reliably extrapolated for the treatment of these conditions from such animal models.

Those skilled in the art will know how to determine efficacy or amounts of a compound to administer based on the results of routine tests in a relevant animal model. The amount of a compound to be administered can be determined in a clinical setting as well based on the response in a treated individual. Modulation of efficacy will depend on the pathological condition and the extent to which progression of apoptosis is desired for treatment or reduction in the severity of the pathological condition. Modulation can be accomplished by adjusting the particular compound, formulation, or dosing strategy. Based on the guidance provided herein and what is well known in the art, those skilled in the art will be able to modulate efficacy in response to well known indicators of the severity of the particular condition being treated. For a description of indicators for the various pathological conditions described herein or otherwise known to be characterized by a pathologically reduced level of apoptosis see, for example, The Merck Manual, Sixteenth Ed, (Berkow, R., Editor) Rahway, N.J., 1992.

By virtue of the cytopathic effect on individual cells, in some embodiments the inventive method can reduce or substantially eliminate the number of cells added to the tumor mass over time. Preferably, the inventive method effects a reduction in the number of cells within a tumor, and, most preferably, the method leads to the partial or complete destruction of the tumor (e.g., via killing a portion or substantially all of the cells within the tumor).

Where the targeted cell is associated with a neoplastic disorder within a patient (e.g., a human), some embodiments of the invention provide a method of treating the patient by first administering a pro-apoptotic modulator of an anti-apoptotic Bcl-2 polypeptide to the patient (“pretreatment”) and subsequently administering a cytotoxic agent to the patient. This approach is effective in treating mammals bearing intact or disseminated cancer. For example, where the cells are disseminated cells (e.g., metastatic neoplasia), the cytopathic effects of the inventive method can reduce or substantially eliminate the potential for further spread of neoplastic cells throughout the patient, thereby also reducing or minimizing the probability that such cells will proliferate to form novel tumors within the patient. Furthermore, by retarding the growth of tumors including neoplastic cells, the inventive method reduces the likelihood that cells from such tumors will eventually metastasize or disseminate. As will be appreciated by one of skill in the art, when the inventive method achieves actual reduction in tumor size (and especially elimination of the tumor), the method attenuates the pathogenic effects of such tumors within the patient. Another exemplary application is in high-dose chemotherapy requiring bone marrow transplant or reconstruction (e.g., to treat leukemic disorders) to reduce the likelihood that neoplastic cells will persist or successfully regrow.

In many instances, the pretreatment of cells or tumors with a pro-apoptotic modulator of an anti-apoptotic Bcl-2 polypeptide before treatment with the cytotoxic agent effects an additive and often synergistic degree of cell death. In this context, if the effect of two compounds administered together in vitro (at a given concentration) is greater than the sum of the effects of each compound administered individually (at the same concentration), then the two compounds are considered to act synergistically. Such synergy is often achieved with cytotoxic agents able to act against cells in the Go-G₁ phase of the cell cycle.

### Ex Vivo Applications

Compounds disclosed herein, for example, compounds that inhibit binding of anti-apoptotic Bcl-2 polypeptides to BH3 domain-containing proteins, can be used in ex vivo applications, for example, bioproduction and cell preservation. One embodiment of the invention additionally provides compounds and methods for inhibiting apoptosis by promoting the anti-apoptotic activity of anti-apoptotic Bcl-2 polypeptides. Compounds that compete for BH3 binding, rather than mimicking the activity of a BH3 domain protein as an endogenous antagonist, can compete for BH3 domain binding, thereby inhibiting the BH3 domain antagonist activity and functioning as an agonist of the anti-apoptotic activity of an anti-apoptotic Bcl-2 polypeptide. Therefore, one embodiment of the invention additionally provides methods of screening compounds and compounds identified by the methods having activity that promotes the anti-apoptotic activity of an anti-apoptotic Bcl-2 polypeptide. Methods for identifying compounds that increase or decrease the anti-apoptotic activity of an anti-apoptotic Bcl-2 polypeptide are well known to those skilled in the art, as disclosed herein.

Thus, in some embodiments, the compounds disclosed herein can be used in ex vivo methods to inhibit apoptosis, thereby promoting cell preservation. Exemplary ex vivo applications of the compounds disclosed herein include, but are not limited to, blood banking; in vitro fertilization, for example, egg preservation or sperm preservation, both for human and veterinary applications; stem cell based products, including embryonic, fetal and adult stem cells; hybridomas for monoclonal antibody production; genetically engineered cells producing recombinant proteins; skin grafts; organ preservation for allograft and transplantation, and the like. Various embodiments of the invention thus additionally provide methods of promoting cell preservation using the compounds disclosed herein.

In other embodiments, methods are provided for reducing the ability of a cell to survive ex vivo. The methods can include the steps of contacting a cell ex vivo with a compound, wherein the compound inhibits binding of an anti-apoptotic Bcl-2 polypeptide to a BH3 domain and increases apoptosis in the cell. The cell can be contacted with the compound using the methods described herein for promoting or increasing apoptosis in a cell. The methods can be used to induce apoptosis and remove a particular subpopulation of cells in a sample containing a population of cells using the targeting methods described herein, such as the attachment of a targeting moiety to the compound.

### Screening of Compounds

A variety of assays are well known in the art that can be used to identify a compound that inhibits the activity of an anti-apoptotic Bcl-2 polypeptide such as, for example, binding to a BH3 domain. Such methods include binding assays where candidate binding compounds are added to a complex that contains an anti-apoptotic Bcl-2 polypeptide and can include, for example, a BH3 domain if being screened for inhibitory activity of binding to a BH3 domain. The anti-apoptotic Bcl-2 polypeptide and/or BH3 domain can be immobilized, for example, to a latex bead, plate or other solid support, or can be free in solution. The anti-apoptotic Bcl-2 polypeptide, BH3 domain or candidate binding compound can be conjugated to a radiolabel, fluorescent label or enzyme label such as alkaline phosphatase, horse radish peroxidase, luciferase, and the like.

In some embodiments, methods are provided for identifying an anti-apoptotic Bcl-2 polypeptide binding compound by contacting an anti-apoptotic Bcl-2 polypeptide with a candidate binding compound in the presence of a compound labeled with a detectable moiety, wherein the labeled compound is selected from a compound as disclosed herein, for example a compound shown Tables 2, 6-8, or derived from a core structure disclosed herein; and measuring the binding of the labeled compound to the anti-apoptotic Bcl-2 polypeptide, wherein a decrease in binding of the labeled compound in the presence of the candidate compound relative to the absence of the candidate compound identifies an anti-apoptotic Bcl-2 polypeptide binding compound. The detectable moiety can be, for example, a fluorophore. In an additional embodiment, the method can include the step of determining the activity of the compound, wherein a compound that increases the anti-apoptotic activity of an anti-apoptotic Bcl-2 polypeptide is an agonist of the anti-apoptotic activity, and wherein a compound that decreases the anti-apoptotic activity is an antagonist. Methods for determining the activity of a compound as an agonist or antagonist are well known in the art and can be determined using routine screening assays and measuring an increase or decrease in apoptosis, as disclosed herein. It is understood by one skilled in the art that appropriate negative and positive controls can be used to determine the relative activity of a compound. For example, a negative control can be an amount of solvent equal to that contributed by a candidate compound in an assay or can be a compound known to not have activity.

In some embodiments, fluorescence-based assays can be used for identifying a compound that inhibits an anti-apoptotic Bcl-2 polypeptide activity such as binding to a BH3 domain (see Examples). Examples of fluorescence methods applicable to such a compound include observations of fluorescence intensity changes resulting from an alteration in interaction between compound and target; fluorescence resonance energy transfer (FRET), which is useful for determining a change in fluorescence intensity based on distance between compound and target; fluorescence polarization changes resulting in a change in size of an observed binding partner when associated or dissociated from another binding partner; fluorescence lifetime changes, and fluorescence correlation spectroscopy, which is based on translation diffusion, a parameter related to the size of an observed binding partner. Such methods can involve employing a fluorescently labeled agent or binding partner. For example, a fluorophore can be detected based on the excitation or emission wavelengths of the fluorophore, fluorescence polarization of the fluorophore, or intensity of fluorescence emitted from the fluorophore. In some embodiments, detection can be based on a difference in a measurable property of the label for the bound and unbound state. For example, in the fluorescence polarization assay (FPA), a difference in fluorescence polarization due to the slower rotation of a molecule such as a BH3 domain or polypeptide containing a BH3 domain bound to an anti-apoptotic Bcl-2 polypeptide compared to the unbound form can be used to detect association. FPA is described in more detail below.

Other measurable differences that can be used to determine association of a fluorophore-labeled compound with an anti-apoptotic Bcl-2 polypeptide include, for example, different emission intensity due to the presence or absence of a quenching agent, difference in emission wavelength due to the presence or absence of a fluorescence resonance energy transfer (FRET) donor or acceptor, or difference in emission wavelength due to differences in fluorophore conformation or environment. An anti-apoptotic Bcl-2 polypeptide or BH3 domain used in a method disclosed herein can be labeled with any of a variety of labels including, for example, those described herein such as fluorescent proteins (see Examples) or the attachment of fluorophores (Hermanson, Bioconjugate Techniques, Academic Press, San Diego (1996)). A labeled BH3 domain that is bound to an anti-apoptotic Bcl-2 polypeptide can be detected according to a known measurable property of the label. It is understood that, while exemplified with a labeled BH3 domain, a labeled anti-apoptotic Bcl-2 polypeptide can also be used in appropriate assay formats, as desired and as described herein.

In some embodiments, FPAs using a fluorescently-labeled, known binder of an anti-apoptotic Bcl-2 polypeptide can be used for identifying a compound that inhibits an anti-apoptotic Bcl-2 polypeptide activity such as binding to a BH3 domain. In some embodiments, a known molecule that binds Bfl-1 (e.g., (FITC)-Bid BH3 peptide) is fluorescently labeled and incubated with Bfl-1 in the presence or absence of a candidate binding compound, or library of compounds, followed by determination of the resulting level of Bfl-1 polarization. If the polarization of the Bfl-1 in the presence of the candidate binding compound is significantly less than in the absence of the candidate binding compound, then the candidate binding compound may be capable of modulating the activity of Bfl-1. By “significantly less”, it is meant that the amount of fluorescence polarization observed in the presence of the candidate binding compound is about 99% less, 95% less, 90% less, 85% less, 80% less, 75% less, 70% less, 65% less, 60% less, 55% less, 50% less, 45% less, 40% less, 35% less, 30% less, 25% less or 20% less than the fluorescence polarization observed in the absence of the candidate binding compound. In one embodiment, the amount of fluorescence polarization observed in the presence of the candidate binding compound is about 50% of the fluorescence polarization observed in the presence of the candidate binding compound. Once such competitive inhibitors are identified, their ability to promote apoptosis in transformed cell lines is confirmed using cellular apoptosis assays well known in the art, such as those described herein.

When a candidate binding compound, or library of compounds, is screened using this assay in, for example, a 96-well format or 384-well (or greater) high throughput format, compounds that have minimal effect on the interaction of an anti-apoptotic Bcl-2 polypeptide exhibit high levels of polarization because very little of the fluorescently-labeled inhibitor is displaced from the anti-apoptotic Bcl-2 polypeptide. In contrast, competitive inhibitors result in reduced polarization due to competitive displacement of the fluorescently-labeled inhibitor from the anti-apoptotic Bcl-2 polypeptide and replacement by the non-labeled competitive inhibitor.

Although the FPA assay is exemplified herein using certain anti-apoptotic Bcl-2 polypeptides and inhibitors, it will be appreciated that any member of the Bcl-2 family of proteins (e.g., Bcl-2, Bcl-X_{L}, Mcl-1, Bfl-1 (A1), Bcl-W and Bcl-B), and any fluorescently labeled inhibitor known to bind to these proteins, can be used within the assay described herein. Although fluorescein isothiocyanate (FITC)-labeled peptide inhibitors are exemplified herein, other fluorescent labels may also be used, including Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

In some embodiments of the FPA, the anti-apoptotic Bcl-2 polypeptide can be incubated in the presence of a candidate binding compound, then incubated with a labeled known inhibitor to reduce time-dependent anti-apoptotic Bcl-2 polypeptide to the labeled known inhibitor. For example, the anti-apoptotic Bcl-2 polypeptide can be incubated in the presences of a candidate binding compound for about 15 minutes to about three hours. In some embodiments, the anti-apoptotic Bcl-2 polypeptide can be incubated in the presence of a candidate binding compound for about 30 minutes to about one hour. In some embodiments, the anti-apoptotic Bcl-2 polypeptide can be incubated in the presence of a candidate binding compound for about one hour, and then incubated for about 4 hours after the subsequent addition of labeled known inhibitor. In other embodiments, the anti-apoptotic Bcl-2 polypeptide can be incubated with the labeled known inhibitor from about 30 minutes to about 10 hours.

In some embodiments, the FPA used to identify compounds that bind to the Bcl-2-family member involves preparing a first reaction mixture comprising an anti-apoptotic Bcl-2 polypeptide, fluorescently labeled inhibitor and candidate binding compound, and a second mixture comprising the same anti-apoptotic Bcl-2 polypeptide and fluorescently labeled inhibitor under conditions and for a time sufficient to allow the components to interact and bind, thus forming a complex which can be removed and/or detected in the reaction mixture.

In some embodiments, an FPA can be used to identify inhibitors of an anti-apoptotic Bcl-2 polypeptide as follows: a) an anti-apoptotic Bcl-2 polypeptide is anchored to a solid phase; b) immobilized anti-apoptotic Bcl-2 polypeptide is incubated with a known inhibitor peptide labeled with a fluorescent tag or other reporter molecule, in the presence or absence of compounds being tested; c) after incubation under suitable conditions, the solid phase is washed to remove unbound reactants; d) the amount of labeled inhibitor peptide bound to the solid phase is measured for each reaction; and e) the amount of labeled inhibitor peptide bound in the presence of various candidate binding compounds is compared with the amount of labeled inhibitor peptide bound in the absence of candidate binding compounds, and the ability of each candidate binding compound to compete for anti-apoptotic Bcl-2 polypeptide binding sites is determined.

In practice, microtiter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In some embodiments, the nonimmobilized component can be added to the coated surface containing the anchored component. After the reaction is complete, unreacted components can be removed (e.g., by washing) under conditions such that any complexes formed will remain immobilized on the solid surface. The detection of complexes anchored on the solid surface can be accomplished in a number of ways. Where the previously nonimmobilized component can be pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, can be directly labeled or indirectly labeled with a labeled anti-Ig antibody).

In some embodiments, a reaction can be conducted in a liquid phase, the reaction products separated from unreacted components, and complexes detected, e.g., using an immobilized antibody specific for the Bcl related protein, polypeptide, peptide or fusion protein or the candidate binding compound to anchor any complexes formed in solution, and a labeled antibody specific for the other component of the possible complex to detect anchored complexes.

In some embodiments, cell-based assays can be used to identify compounds that interact with an anti-apoptotic Bcl-2 polypeptide or compounds that enhance or inhibit the interaction of an anti-apoptotic Bcl-2 polypeptide with inhibitor peptide. In some embodiments, cell lines that express an anti-apoptotic Bcl-2 polypeptide, or cell lines (e.g., COS cells, CHO cells, fibroblasts, etc.) that have been genetically engineered to express an anti-apoptotic Bcl-2 polypeptide (e.g., by transfection or transduction of DNA) can be used.

One skilled in the art understands that a variety of additional assays can be used to determine whether a candidate binding compound is a compound that inhibits an anti-apoptotic Bcl-2 polypeptide activity such as binding to a BH3 domain. For example, a scintillation proximity assay (Alouani, Methods Mol. Biol. 138:135-41 (2000)) can be used. Scintillation proximity assays involve the use of a fluomicrosphere coated with an acceptor molecule, such as an antibody, to which an antigen will bind selectively in a reversible manner. For example, an anti-apoptotic Bcl-2 polypeptide/BH3 domain complex can be bound to a fluomicrosphere using an antibody that specifically binds to the anti-apoptotic Bcl-2 polypeptide, and contacted with a 3H or 125I labeled candidate binding compound. If the labeled candidate agent specifically binds to the anti-apoptotic Bcl-2 polypeptide, the radiation energy from the labeled candidate binding compound is absorbed by the fluomicrosphere, thereby producing light which is easily measured.

Additional assays suitable for identifying a compound that inhibits an anti-apoptotic Bcl-2 polypeptide activity such as binding to a BH3 domain can include, without limitation, UV or chemical cross-linking assays (Fancy, Curr. Opin. Chem. Biol. 4:28-33 (2000)) and biomolecular interaction analyses (Weinberger et al., Pharmacogenomics 1:395-416 (2000)). Specific binding of a candidate binding compound to an anti-apoptotic Bcl-2 polypeptide can be determined by cross-linking these two components, if they are in contact with each other, using UV or a chemical cross-linking agent. In addition, a biomolecular interaction analysis (BIA) can detect whether two components are in contact with each other. In such an assay, one component, such as an anti-apoptotic Bcl-2 polypeptide/BH3 domain complex is bound to a BIA chip, and a second component such as a candidate compound is passed over the chip. If the candidate compound displaces the anti-apoptotic Bcl-2 polypeptide or BH3 domain by binding to the anti-apoptotic Bcl-2, the contact results in an electrical signal, which is readily detected.

Further assays suitable for identifying a compound that inhibits an anti-apoptotic Bcl-2 polypeptide activity such as binding to a BH3 domain include those based on NMR methods. Such methods take advantage of the significant perturbations that can be observed in NMR-sensitive parameters of a candidate compound or its target, such as an anti-apoptotic Bcl-2 polypeptide or domain thereof, that occur upon complex formation between the compound and target. These perturbations can be used to detect binding between a candidate binding compound and an anti-apoptotic Bcl-2 polypeptide, as well as to assess the strength of the binding interaction. In addition, some NMR techniques allow the identification of the compound binding site or which part of the compound is responsible for interacting with the target. Exemplary NMR methods useful for identifying a compound that inhibits an anti-apoptotic Bcl-2 polypeptide activity such as binding to a BH3 domain include “SAR by NMR,” which is described, for example, in Shuker et al. Science 274:1531-1534 (1996), and a variety of NMR-based screening assays, including SHAPES screening, fragment-based approaches for lead optimization using NMR, and fluorine-NMR competition binding experiments, all of which are described, for example, in Combinatorial Chemistry & High Throughput Screening, Vol. 5, No. 8 (2002), and in Hajduk et al., Quarterly Reviews of Biophysics 32(3):211-240 (1999).

Dissociation of the labeled binding partner such as a BH3 domain can be detected as absence or reduction in the amount of label from an anti-apoptotic Bcl-2 polypeptide in the presence of a competitive binding candidate compound or as a reversal of a change that occurs upon association of the labeled binding partner such as a BH3 domain with an anti-apoptotic Bcl-2 polypeptide in the presence of a competitive binding candidate compound. Thus, dissociation can be detected in the presence of a non-labeled candidate binding compound as a reduction or loss of radioactivity of the anti-apoptotic Bcl-2 polypeptide in the presence of a radionucloetide labeled binding partner such as a BH3 domain, reduction or loss of electromagnetic absorbance at a specified wavelength for the anti-apoptotic Bcl-2 polypeptide in the presence of a chromophore labeled binding partner such as a BH3 domain, reduction or loss of magnetic signal at a specified field strength or radio frequency for the anti-apoptotic Bcl-2 polypeptide in the presence of a paramagnetic spin labeled binding partner such as a BH3 domain, or reduction or loss of a secondary label associated with the anti-apoptotic Bcl-2 polypeptide in the presence of a binding partner such as a BH3 domain that is labeled with a binding group for the secondary label.

Other changes in a property of a label that can be detected to determine association or dissociation of an appropriately labeled binding partner such as a BH3 domain and an anti-apoptotic Bcl-2 polypeptide include, for example, absorption and emission of heat, absorption and emission of electromagnetic radiation, affinity for a receptor, molecular weight, density, mass, electric charge, conductivity, magnetic moment of nuclei, spin state of electrons, polarity, molecular shape, or molecular size. Properties of the surrounding environment that can change upon association or dissociation of an appropriately labeled binding partner such as a BH3 domain and an anti-apoptotic Bcl-2 polypeptide include, for example, temperature and refractive index of surrounding solvent. Association and dissociation of a binding partner such as a BH3 domain from an anti-apoptotic Bcl-2 polypeptide can be measured based on any of a variety of properties of a labeled binding partner or of the complex between a binding partner and an anti-apoptotic Bcl-2 polypeptide using well known methods including, for example, equilibrium binding analysis, competition assays, and kinetic assays as described in Segel, Enzyme Kinetics, John Wiley and Sons, New York (1975), and Kyte, Mechanism in Protein Chemistry, Garland Pub. (1995).

In addition, virtual computational methods and the like can be used to identify compounds that can displace a binding partner such as a BH3 domain from an anti-apoptotic Bcl-2 polypeptide in a screening method disclosed herein. Exemplary virtual computational methodology involves virtual docking of small-molecule agents on a virtual representation of an anti-apoptotic Bcl-2 polypeptide or an anti-apoptotic Bcl-2 polypeptide/BH3 domain complex structure in order to determine or predict specific binding (see, for example, Shukur et al., supra, 1996; Lengauer et al., Current Opin. Struct. Biol. 6:402-406 (1996); Choichet et al., J. Mol. Biol. 221:327-346 (1991); Cherfils et al., Proteins 11:271-280 (1991); Palma et al., Proteins 39:372-384 (2000); Eckert et al., Cell 99:103-115 (1999); Loo et al., Med. Res. Rev. 19:307-319 (1999); Kramer et al., J. Biol. Chem. 276: 7291-7301 (2001)).

The methods disclosed herein for identifying a compound that inhibits an anti-apoptotic Bcl-2 polypeptide activity such as binding to a BH3 domain can be performed using low throughput or high throughput assay formats. Screening can be carried out in all plate formats, including for example, 96, 384 and 1536 well formats. In addition, assays such as those described above can be performed in kinetic-based or end point-based formats. To increase screening throughout, more than one candidate binding compound can be present in an assay sample. The number of different candidate compounds to test in the methods disclosed herein will depend on the application of the method. For example, one or a small number of candidate compounds can be screened using manual screening procedures, or when it is desired to compare efficacy among several candidate agents. However, it will be appreciated that the larger the number of candidate compounds, the greater the likelihood of identifying a compound having the desired activity in a screening assay. Additionally, large numbers of candidate compounds can be processed in high-throughput automated screening methods.

In some embodiments, methods are provided for identifying a compound that inhibits the activity of an anti-apoptotic Bcl-2 polypeptide in a database. A database of molecules such as peptides or small molecules can be queried with the structure of an anti-apoptotic Bcl-2 polypeptide to identify candidate agents having a moiety identical or similar to the query structure. A candidate compound identified in a database search can be synthesized, isolated or otherwise obtained using known methods and then tested for its level of activity as an inhibitor of an anti-apoptotic Bcl-2 polypeptide activity using the assays described herein.

### Assay Development

The compounds disclosed herein can also be used to develop assays. A compound that binds to an anti-apoptotic Bcl-2 polypeptide can be used to identify additional compounds that compete for binding of a compound having anti-apoptotic Bcl-2 polypeptide binding activity. For example, a compound that binds to an anti-apoptotic Bcl-2 polypeptide, such as those shown in Tables 2, 6-8, or in core structures I-IV, can be conjugated to a fluorophore or other detectable moiety, as disclosed herein. The labeled compound can be used to screen for additional binding compounds that compete for binding of or displace the labeled compound. Fluorescently labeled compounds are particularly useful in high throughput screening (HTS) applications.

### Compound Libraries and Preparation of Compounds

Compounds can be identified, as disclosed herein, by screening a library of compounds, including commercially available libraries or publicly available libraries, or compound libraries can be synthesized using well known methods, including combinatorial chemical libraries (see, for example, Mendonca and Xiao, Med. Res. Rev. 19:451-462 (1999); van Maarseveen, Comb. Chem. High Throughput Screen. 1:185-214 (1998); Andres et al., Comb. Chem. High Throughput Screen. 2:191-210 (1999); Sucholeiki, Mol. Divers. 4:25-30 (1998-1999); Ito and Manabe, Curr. Opin. Chem. Biol. 2:701-708 (1998); Labadie, Curr. Opin. Chem. Biol. 2:346-352 (1998); Backes and Ellman, Curr. Opin. Chem. Biol. 1:86-93 (1997); Kihlberg et al., Methods Enzymol. 289:221-245 (1997); Blackburn and Kates, Methods Enzymol. 289:175-198 (1997); Meldal, Methods Enzymol. 289:83-104 (1997); Merrifield, Methods Enzymol. 289:3-13 (1997); Thuong and Asseline, Biochimie. 67:673-684 (1985)).

Computer modeling and searching technologies permit identification of compounds, or the improvement of already identified compounds that can modulate anti-apoptotic Bcl-2 polypeptides. Having identified such a compound or composition, the active sites or regions are identified. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures can be measured with a complexed ligand, natural or artificial, which can increase the accuracy of the active site structure determined.

If an incomplete or insufficiently accurate structure is determined, the methods of computer based numerical modeling can be used to complete the structure or improve its accuracy. Any recognized modeling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential anti-apoptotic Bcl-2 polypeptide binding compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of anti-apoptotic Bcl-2 polypeptides will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen et al. 1988 Acta Pharm Fennica 97:159-166; McKinaly and Rossmann 1989 Annu Rev Pharmacol Toxicol 29:111-122; Perry and Davies, OSAR: Quantitative Structure-Activity Relationships in Drug Design, pp. 189-193, Alan R. Liss, Inc. (1989); Lewis and Dean 1989 Proc R Soc Lond 236:125-140 and 141-162; and, with respect to a model receptor for nucleic acid components, Askew et al. 1989 J Am Chem Soc 111:1082-1090. Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario).

### Structure-Based Drug Design

To aid in the characterization and optimization of compounds that can alter the activity of anti-apoptotic Bcl-2 polypeptides, structure-based drug design has become a useful tool. Solution nuclear magnetic resonance (NMR) techniques can be used to map the interactions between the BH3 domain of the anti-apoptotic Bcl-2 polypeptide and chemical compounds that target these anti-apoptotic polypeptides. NMR chemical shift perturbation is an efficient tool for rapid mapping of interaction interfaces on proteins. Structure-activity relationships (SAR) can be obtained by using nuclear magnetic resonance (NMR), using the method known as “SAR by NMR” (Shuker et al. 1996 Science 274:1531; Lugovskoy et al. 2002 J Am Chem Soc 124:1234). SAR by NMR can be used to identify, optimize and link together small organic molecules that bind to proximal subsites of a protein to produce high-affinity ligands.

In using NMR to structurally characterize protein-protein and ligand-protein interactions, isotope labeling can result in increased sensitivity and resolution, and in reduced complexity of the NMR spectra. The three most commonly used stable isotopes for macromolecular NMR are ¹³C, ¹⁵N and ²H. Isotope labeling has enabled the efficient use of heteronuclear multi-dimensional NMR experiments, providing alternative approaches to the spectral assignment process and additional structural constraints from spin-spin coupling. Uniform isotope labeling of the protein enables the assignment process through sequential assignment with multidimensional triple-resonance experiments and supports the collection of conformational constraints in de novo protein structure determinations (Kay et al. 1990 J Magn Reson 89:496; Kay et al. 1997 Curr Opin Struct Biol 7:722). These assignments can be used to map the interactions of a ligand by following chemical-shift changes upon ligand binding. In addition, intermolecular NOE (nuclear Overhauser effect) derived inter-molecular distances can be obtained to structurally characterize protein-ligand complexes.

In addition to uniform labeling, selective labeling of individual amino acids or labeling of only certain types of amino acids in proteins can result in a dramatic simplification of the spectrum and, in certain cases, enable the study of significantly larger macromolecules. For example, the methyl groups of certain amino acids can be specifically labeled with ¹³C and ¹H in an otherwise fully ²H-labeled protein. This results in well resolved heteronuclear [¹³C,¹H]-correlation spectra, which enables straightforward ligand-binding studies either by chemical shift mapping or by protein methyl-ligand inter-molecular NOEs, thus providing key information for structure-based drug design in proteins as large as 170 kDa (Pellecchia et al. 2002 Nature Rev Drug Discovery 1:211). 2D [¹³C,¹H]-HMQC (heteronuclear multiple quantum coherence) and ¹³C-edited [¹H,¹H]-NOESY NMR experiments on a ligand-receptor complex can be used to detect binding, determine the dissociation constant for the complex, and provide a low-resolution model based on the available three-dimensional structure of the target, thus revealing the relative position of the ligand with respect to labeled side-chains.

Thus, NMR can be used to identify molecules that induce apoptosis. Compounds can be screened for binding to labeled Bfl-1, for example. Such labels include ¹⁵N and ¹³C. The interaction between the compound and Bfl-1, and therefore its ability to induce apoptosis, are determined via NMR. Accordingly, one embodiment of the invention is a method of optimizing compounds discovered by the methods described herein through NMR analysis. A target compound that is found to affect the binding between Bfl-1 and a compound known to bind to and inhibit Bfl-1 is provided. That target compound is then reacted with a library of chemical fragments in the presence of Bfl-1 in order to determine chemical fragments that bind a site adjacent to the target compound. Chemical fragments discovered to bind a site adjacent to the binding site of the target compound are covalently linked to the target compound to provide an optimized target compound.

### Kits

One embodiment of the invention further provides a kit, including at least one compound that has activity as an inhibitor of anti-apoptotic Bcl-2 polypeptide activity and a second compound having therapeutic activity. A compound that can be included in a kit includes, for example, a compound such as those shown in Tables 2-8 or a core structure disclosed herein. In some embodiments the compound can be MLS-0067130. Such kits are useful, for example, in the treatment of a condition characterized by a pathologically reduced level of apoptosis.

In some embodiments, kits are providing which include compounds as separately packaged formulations or in a mixed formulation, so long as the compounds are provided in an amount sufficient to have a therapeutic effect following at least one administration of each compound. The formulations can be any of those described above, or otherwise known to be appropriate for the particular compound and mode of administration. The contents of a kit disclosed herein are housed in packaging material or other suitable physical structure, preferably to provide a sterile, contaminant-free environment. In addition, the packaging material contains instructions indicating how the materials within the kit can be administered for treatment of a condition characterized by a pathologically reduced level of apoptosis. The instructions for use typically include a tangible expression describing the route of administration or, if required, methods for preparing the formulation for administration. The instructions can also include identification of potential effects from use of the kit's contents or a warning regarding improper use of the contents of the kit.

### EXAMPLES

### Example 1

### Assays for Screening for Inhibitors of Anti-Apoptotic Bcl-2 Polypeptides

To identify and optimize chemical inhibitors of Bfl-1, procedures were devised for producing multi-milligram quantities of purified recombinant Bfl-1 protein. A fluorescence polarization assay (FPA), using a Bfl-1-binding synthetic peptide conjugated with fluorescein isothiocyanate (FITC), was also devised. A preliminary screen was performed on approximately 10,000 compounds, demonstrating the suitability of the assay for the high-throughput screening.

To identify compounds selective for Bfl-1, FPAs were also developed for each of the other five anti-apoptotic members of the mammalian Bcl-2 family, Bcl-2, Bcl-XL, Mcl-1, Bcl-W, and Bcl-B. Using such assays, compounds can be screened that inhibit Bfl-1 but not other members of the Bcl-2-family, or that inhibit all members or a subset of members of the Bcl-2 family, providing chemical inhibitors specific for Bfl-1, subsets of the Bcl-2 family, and/or all Bcl-2 members. A systemic analysis of previously known chemical inhibitors of Bcl-2 thus far described in the literature was performed (Zhai et al., Cell Death Diff. 13:1419-1421 (2006)), using FPAs for each of the six anti-apoptotic members of the Bcl-2 family. None of the synthetic compounds or natural products previously reported selectively binds Bfl-1 with a biologically relevant affinity, based on competition assays using FITC-BH3 peptides. Several compounds bind the various members of the Bcl-2 family with affinities in the 0.1-3 μM range, and some bind selectively to certain subsets of the Bcl-2 family (Oltersdorf et al., Nature 435:677-681 (2005)). Thus, the pocket on Bfl-1 that binds BH3 peptides appears to be sufficiently different from other Bcl-2 family members such that selective inhibitors can be identified, either directly from screens of diverse libraries or secondarily through generating chemical analogs of compounds that interact with Bfl-1.

The generation of reagents and assays are described below in more detail.

Protein purification. GST-fusion proteins containing Bcl-XL, Bcl-2, Bcl-W, Bcl-B, Bfl-1 and Mcl-1 lacking their C-terminal transmembrane domains (approximately last 20 amino-acids) (“ΔTM”) were expressed from the pGEX 4T-1 plasmid in XL-1 Blue cells (Stratagene, Inc., San Diego Calif.). Briefly, cells were grown in 2 L of Luria Broth (LB) with 50 μg/mL ampicillin at 37° C. to an OD600 nm of 1.0., then isopropyl-beta-D-thiogalactopyranoside (IPTG) (0.5 M) was added, and the cultures were incubated at 25° C. for 6 h. Cells were then recovered in 20 mM phosphate buffer (pH 7.4), 150 mM NaCl, 1 mM dithiotthreitol (DTT), 1 mM ethylenediaminetetraacetic acid (EDTA), 1 mM phenylmethylsulfonyl fluoride (PMSF), followed by sonication. Cellular debris were sedimented by centrifugation at 27,500×g for 20 min, and the resulting supernatants were incubated with 10 mL of Glutathionine-Sepharose™ (Pharmacia/Amersham/GE Healthcare; Piscataway N.J.) at 4° C. for 2 h. The resin was washed 3 times with 20 mM phosphate buffer (pH 7.4), 150 mM NaCl, and 1 mM DTT, and then 10 mM of reduced glutathione dissolved in 50 mM Tris-HCl (pH 8.0) was used to elute the GST-fusion proteins.

Fluorescence polarization assays (FPA). To determine the binding affinity of anti-apoptotic Bcl-2 polypeptides to FITC-conjugated Bid BH3 peptide, fluorescence polarization assays (FPAs) were performed according to published procedures (Zhai et al., Biochem. J. 376:229-236 (2003)). Briefly, a serial concentration of anti-apoptotic Bcl-2 polypeptides were incubated with 5 nM of FITC-conjugated synthetic Bid BH3 peptide (FITC-Ahx-EDIIRNIARHLAQVGDSMDR; SEQ ID NO: 1) in phosphate buffered saline (PBS) using 96 well black plates (Greiner bio-one; Monroe N.C.). Fluorescence polarization was measured after 10 min using an Analyst™ AD Assay Detection System (LJL Biosystem, Sunnyvale, Calif.) in phosphate-buffered saline (PBS) (pH 7.4). The IC₅₀ determinations were performed using GraphPad Prism software (GraphPad, Inc., San Diego, Calif.).

Competition assays. Using the same procedure outlined above, 100 nM of GST-Bfl-1 protein were incubated with the compounds at 50 μM concentration for 30 min. Then, 5 nM of FITC-Bid BH3 peptide was added. Fluorescence polarization was measured after 10 min.

Screening Protocol. The FPA has been formatted for 96 well plates, using 100 μl per well final volume. First, 100 nM of GST-Bfl-1 protein (final concentration in PBS (pH 7.4)) was added to 96 well black plates (Greiner bio-one) in a volume of 45 μl. Second, 5 μl of compounds in dimethylsulfoxide (DMSO) were added per well, which results in a final approximate concentration of 50 μM and achieves a final DMSO concentration of 5% when the reactions are subsequently brought to full volume (100 μL). Third, after a 30 min incubation at room temperature, 50 μl of FITC-Bid BH3 peptide was added and plates were incubated at room temperature for 10 min. Fourth, fluorescence polarization was measured using an Analyst™ AD Assay Detection System (LJL Biosystem, Sunnyvale, Calif.) in phosphate-buffered saline (PBS) (pH 7.4). Compounds reducing FP by 50% were considered “hits.”

Prior to large-volume screening, Bfl-1 FPA was further optimized to ensure the assay compatibility with high throughput screening (HTS). In brief, a panel of buffers was tested and optimal buffer was identified. This buffer, 20 mM Bis-Tris-HCl, pH 7.0, containing 0.8 mM Tris(2-carboxyethyl) phosphine (TCEP), 0.004% Tween 20, provided superior stability of the signal and an extended assay window. In addition, the affinity of Bfl-1/FITC-Bid complex was significantly improved, resulting in a substantial decrease of Bfl-1 concentration. An improved assay window showed the feasibility of screening at the concentration of Bfl-1 corresponding to the Kd value of the complex, as opposed to the concentration several fold higher than the Kd employed in FPA in PBS buffer. These changes significantly improved FPA sensitivity and made feasible the development of TR-FRET assays, as discussed below. The decrease in the concentration of Bfl-1 in respect to the concentrations of FITC-Bid and Bfl-1 in the HTS were to 2.2 and 3.0 nM, respectively. During HTS, Bfl-1 was preincubated with the compounds for 1 h at 4° C., then plates were added with FITC-Bid and fluorescence polarization measured after 4 h at room temperature. The signal was stable for 24 h. Fluorescence polarization was measured on an Analyst HT plate reader (Molecular Devices, Inc.; Sunnyvale Calif.) using fluorescein optics. To minimize the number of false positives resulting from fluorescent compounds, the fluorescence intensity of each sample was calculated and normalized to the average fluorescence intensity value of the plate negative control wells to calculate F_ratio parameter. Compounds with F_ratio>1.5 were excluded from further consideration. Compounds were obtained from the NIH Molecular Libraries Small Molecule Repository (MLSMR) (mlsmr.glpg.com/MLSMR_HomePage/), part of the NIH Molecular Libraries Screening Center Network (MLSCN). This compound collection is predominantly composed of commercially available compounds. The information on vendors for various compounds is available on the PubChem database (pubchem.ncbi.nlm.nih.gov). Additional compounds were screened from commercial sources and synthesized chemical libraries.

For the Bfl-1 FPA assay, Bfl-1 protein and FITC-Bid peptide (FITC-Ahx-EDIIRNIARHLAQVGDSMDR) (SEQ ID NO: 1) were utilized. The assay buffer used was 25 mM Bis-Tris, pH 7.0, 1 mM TCEP, 0.005% Tween 20. The Bfl-1 working solution contained 7.4 nM Bfl-1 in assay buffer. The solution was prepared fresh and kept on ice prior to use. The FITC-Bid working solution contained 5.6 nM FITC-Bid in assay buffer.

For the Bfl-1 high throughput screen (HTS), 4 μL of 100 mM compounds in 10% DMSO were dispensed in columns 3-24 of Greiner 384-well black small-volume plates. To columns 1 and 2 were added 4 μL of 10% DMSO. Positive control wells, containing no Bfl-1, were assigned to column 1, and 8 μL of assay buffer were added using WellMate bulk dispenser (Matrix/Thermo Scientific; Hudson N.H.). To columns 2-24, 8 μL of Bfl-1 working solution was added using WellMate bulk dispenser (Matrix). Negative control wells that contained no compounds were assigned to column 2. Plates were incubated for 1 h at 4° C. An aliquot of 8 μL of freshly prepared FITC-Bid working solution was added to the whole plate using WellMate bulk dispenser (Matrix). The final concentrations of the components in the assay were as follows: 20 mM Bis-Tris-HCl, pH 7.0, 0.8 mM TCEP, 0.004% Tween 20; 2.2 nM FITC-Bid (columns 1-24); 3.0 nM Bfl-1 (columns 2-24); 2% DMSO (columns 1-24); and 20 μM compounds (columns 3-24).

Plates were incubated for 4 h at room temperature protected from direct light. Fluorescence polarization was measured on an Analyst HT plate reader (Molecular Devices, Inc) using fluorescein filters: excitation filter −485 nM, emission filter −530 nM, dichroic mirror −505 nM. The signal for each well was acquired for 100 ms. Data analysis was performed using CBIS software (ChemInnovations, Inc). Fluorescence intensity of each sample was normalized to the average fluorescence intensity value of the plate negative control wells to calculate F_ratio parameter.

For the Bfl-1 FPA secondary screening protocol, dose-response curves contained 10 concentrations of compounds obtained using 2-fold serial dilution. Compounds were serially diluted in 100% DMSO, and then diluted with water to 10% final DMSO concentration. Aliquots of 4 μL of compounds in 10% DMSO were transferred into columns 3-22 of Greiner 384-well black small-volume plates. Columns 1-2 and 23-24 contained 4 μL of 10% DMSO. Columns 1-2 were reserved for positive controls, and 8 μL of assay buffer was added using a WellMate bulk dispenser (Matrix). An 8 μL aliquot of Bfl-1 working solution was added to columns 2-24 using a WellMate bulk dispenser (Matrix). Columns 23-24 represented negative control wells. Plates were incubated for 1 h at 4° C. An 8 μL aliquot of freshly prepared FITC-Bid working solution was added to the whole plate using WellMate bulk dispenser (Matrix). Plates were incubated for 4 h at room temperature protected from direct light. Fluorescence polarization was measured on an Analyst HT plate reader (Molecular Devices, Inc) using fluorescein filters: excitation filter −485 nM, emission filter −530 nM, dichroic mirror −505 nM. The signal for each well was acquired for 100 ms. Data analysis was performed using sigmoidal dose-response equation through non-linear regression.

TR-FRET assay. For confirmation of FPA results, a TR-FRET assay utilizing terbium- (Tb-) labeled anti-GST antibody (Th-Ab) was developed. Briefly, a panel of different concentrations of FITC-Bid, Bfl-1 and Tb-Ab were tested, and it was determined that the optimal signal was observed in the FPA buffer in the presence of 2.25 nM FITC-Bid, 2 nM Bfl-1 and 1 nM Tb-Ab. The reaction mixture was preincubated for 1 h, and TR-fluorescence (TRF) was measured on an M5 plate reader (Molecular Devices) using excitation at 340 nm and emission at 520 and 490 nM. The measurements were averaged from 5 readings during 1 ms after an initial delay 0.1 ms. TR-FRET signal was calculated as the ratio of TR-Fluorescence at 520 nm to TR-Fluorescence at 490 mm.

For Bfl-1 TR-FRET assays, Bfl-1 protein and FITC-Bid peptide (FITC-Ahx-EDIIRNIARHLAQVGDSMDR) (SEQ ID NO: 1) were utilized. Terbium-anti-GST antibody (Tb-anti-GST Ab) was obtained from Invitrogen (San Diego Calif.). The assay buffer contained 25 mM Bis-Tris, pH 7.0, 1 mM TCEP, and 0.005% Tween 20. The Tb-Ab buffer contained 2.5 nM Tb-anti-GST antibody in assay buffer. The Bfl-1/Th-Ab working solution contained 5 nM Bfl-1 in Th-Ab assay buffer. The solution was prepared fresh and kept on ice prior to use. The FITC-Bid working solution contained 5.6 nM FITC-Bid in assay buffer.

For the Bfl-1 TR-FRET dose-response screening protocol, dose-response curves contained 10 concentrations of compounds obtained using 2-fold serial dilution. Compounds were serially diluted in 100% DMSO, and then diluted with water to 10% final DMSO concentration. A 4 μl aliquot of compounds in 10% DMSO was transferred into columns 3-22 of Greiner 384-well white small-volume plates. Columns 1-2 and 23-24 contained 4 μL of 10% DMSO.

Columns 1-2 were reserved for positive controls, and 8 μL of Th-Ab buffer was added using a WellMate bulk dispenser (Matrix). An aliquot of 8 μL of Bfl-1/Th-Ab working solution was added to columns 3-24 of Greiner 384 well white plates using a WellMate bulk dispenser (Matrix). Columns 23-24 represented negative control wells. Plates were incubated for 1 h at 4° C. An 8 μL aliquot of freshly prepared FITC-Bid working solution was added to the whole plate using WellMate bulk dispenser (Matrix). Plates were incubated for 4 h at room temperature protected from direct light. Fluorescence was measured on the M5 plate reader (excitation: 340 nm, emission: 490 and 520 nm, cutoff: 475 and 515 nm, respectively) in Time Resolved (TR) mode with signal integrated for 1 ms after initial delay of 0.1 ms. TR-FRET signal was calculated as the ratio of TR-Fluorescence at 520 nm to TR-Fluorescence at 490 nm. Data analysis was performed using a sigmoidal dose-response equation through non-linear regression.

### Example 2

### Screening for Inhibitors of Anti-Apoptotic Bcl-2 Polypeptide Bfl-1

Purification of proteins and assays were performed as described in Example 1.

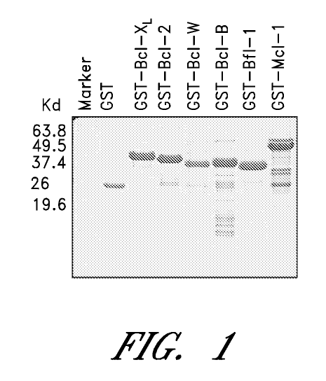

The GST-Bfl-1ΔTM protein was purified. The protein yield for the GST-Bfl-1 protein was approximately 5 mg per liter of cells. 10 μg of each purified protein was analyzed by sodium dodecyl sulfate-polyacrylamide gel electrophoresis (SDS-PAGE) followed by Coomassie Blue staining (FIG. 1). GST protein was used as control. The purity was over 95%, as determined by Coomassie Blue staining of material analyzed by SDS-PAGE. Other anti-apoptotic human Bcl-2 family proteins were also prepared for use in secondary screens of Bfl-1-binding compounds.

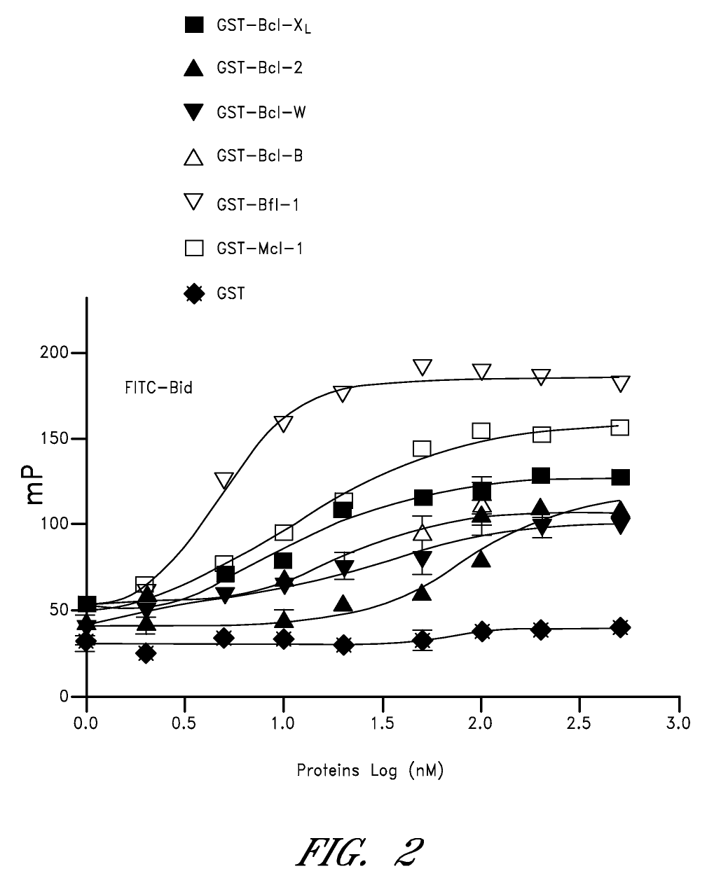

Fluorescence polarization assay (FPA) analysis of Bcl-2-family proteins was performed. Various concentrations of glutathione S transferase (GST) or GST-fusion proteins containing ΔTM versions of Bcl-2, Bcl-X_{L}, Bfl-1, Mcl-1, Bcl-W, and Bcl-B were incubated with 5 nM FITC-conjugated-Bid BH3 peptide in phosphate buffered saline (PBS) (pH 7.4) (FIG. 2). All Bcl-2 family proteins caused fluorescence polarization, but to variable extents, consistent with differences in their individual affinities for this particular BH3 peptide. Fluorescence polarization (in milli-polars) was measured after 10 min. Bfl-1 was found to bind best among the anti-apoptotic Bcl-2 family members to Bid BH3 peptide. The GST control protein did not cause fluorescence polarization (FIG. 2).

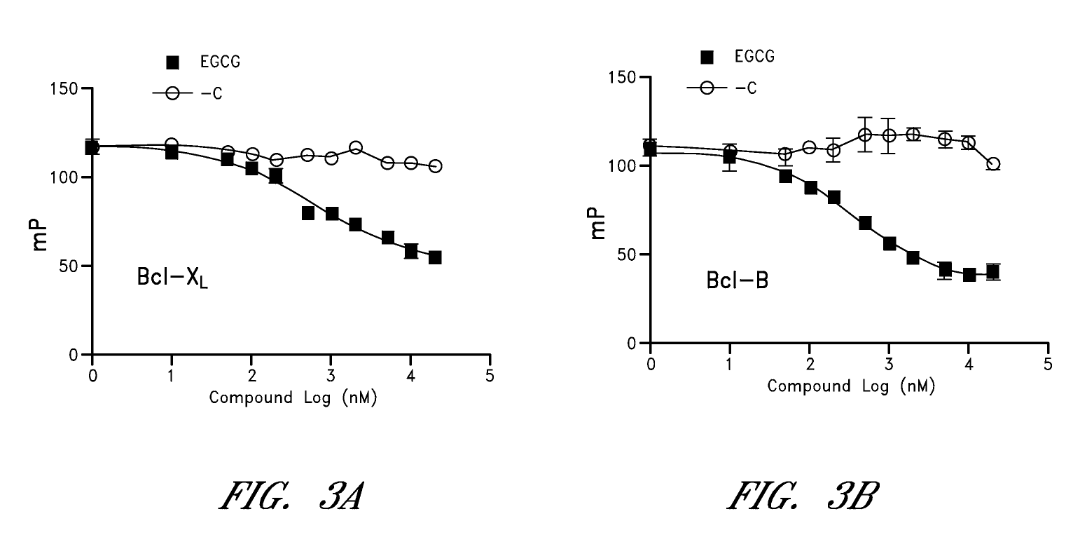

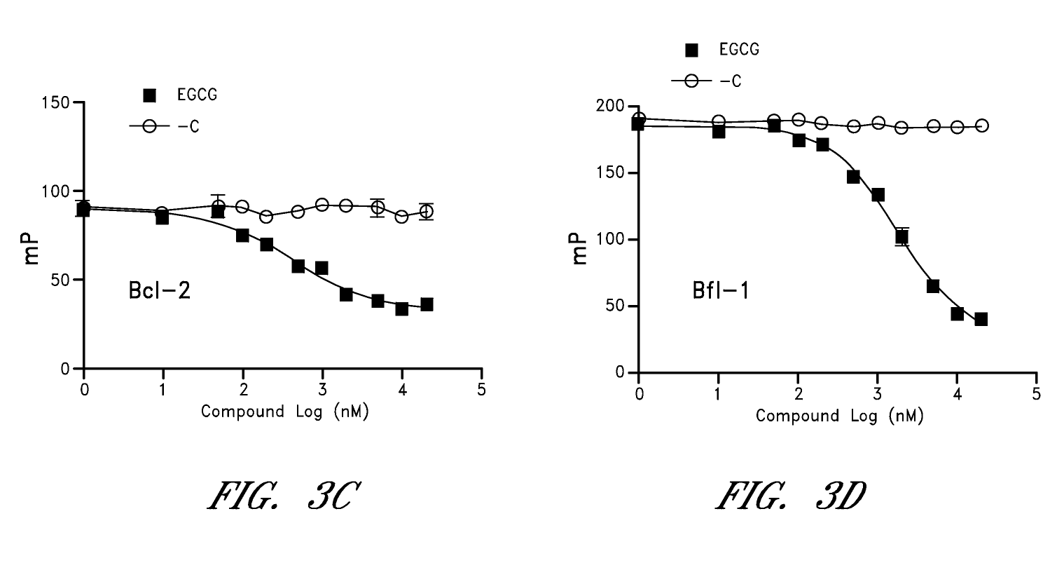

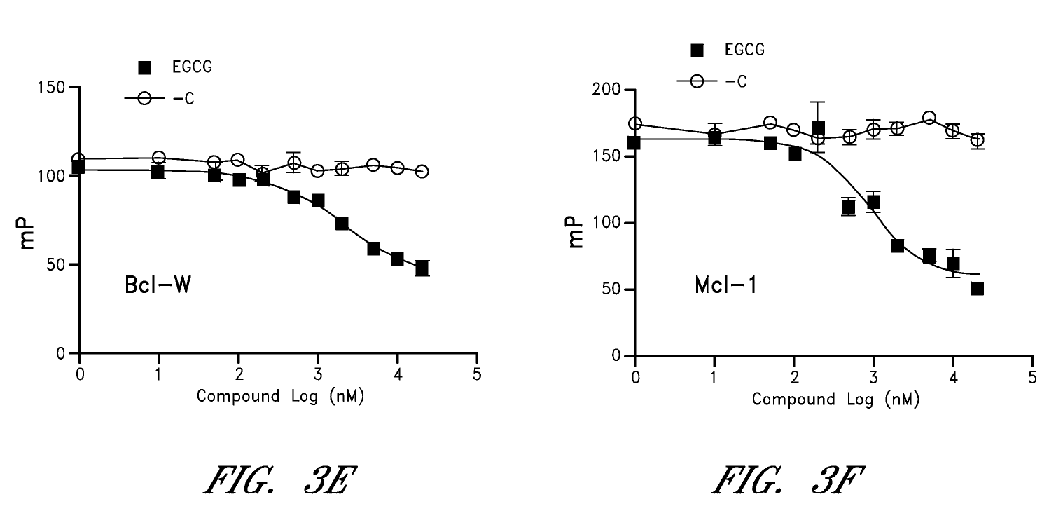

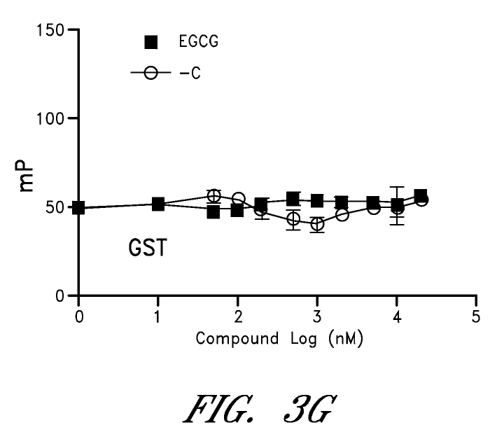

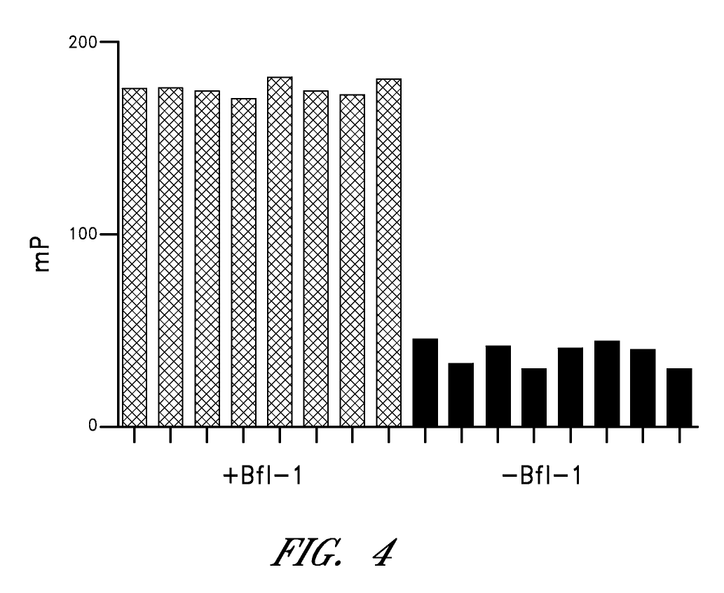

Bcl-2-binding compounds were tested by competition assay analysis. The green tea compound epigallecatechin (EGCG) has been reported to bind Bcl-2 and Bcl-XL (Leone et al., Cancer Res 63:8118-8121 (2003)). The ability of EGCG to compete with fluorochrome-labeled BH3 peptides was confirmed by FPA. 100 nM of GST-Bcl-2 fusion proteins were incubated with various concentrations of EGCG or control compound ECG (“C”) for 2 min in PBS buffer in 50 uL. Next, 5 nM FITC-conjugated-Bid BH3 peptide was added, bringing final volume to 100 uL and final DMSO concentration to 1%. Fluorescence polarization was measured after 20 min. ECGC binds to all six anti-apoptotic members of the Bcl-2-family, to variable extents (FIGS. 3A-G). This compound therefore can serve as a positive control when performing high throughput screens of a chemical library.

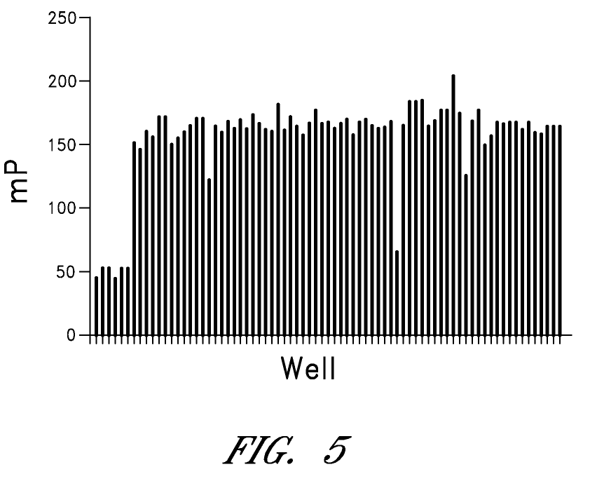

The Z′factor for the Bfl-1 FPA was determined. Eight replicate samples were prepared containing 5 nM FITC-Bid BH3 peptide in PBS. The FITC-conjugated-Bid BH3 peptide was incubated with or without 100 nM GST-Bfl-1 protein in PBS in a total volume of 100 μL in a 96 well black plate. Fluorescence polarization was measured after 10 min. As shown in FIG. 4, assays were performed with (left) or without (right) GST-Bfl-1. FP was measured and the Z′factor was determined to be 0.78. These results show that the Bfl-1 FPA is reproducible.

In a preliminary screen, the Bfl-1 FPA was used to screen a library of 10,000 compounds, representing predominantly natural products. The results from a representative plate in the screen that contained a “hit” are presented in Table 1 (shown below) and FIG. 5. FIG. 5 shows a graphical representation of the data presented in Table 1. In FIG. 5, the Y-axis represents FP in milli-Polars, and the X-axis represents well number (1-96) (A1→H12). The wells A1 to H1 are the negative control (BH3 peptide without GST-Bfl-1 protein) and wells A12 to H12 are the positive control (no compounds). A Bfl-1 inhibitory compound is found in well B9 (candidate hit). From 10,000 compounds, 66 hits were identified. Upon repeat testing, 10 active compounds remained. Thus, the overall hit rate was 0.1%.

TABLE 1

| | 1 | 2 | 3 | 4 | 5 | 6 | 7 | 8 | 9 | 10 | 11 | 12 |
|---|---|---|---|---|---|---|---|---|---|---|---|---|
| A | 43 | 150 | 154 | 158 | 158 | 160 | 154 | 168 | 166 | 158 | 156 | 148 |
| B | 44 | 145 | 159 | 153 | 167 | 159 | 165 | 156 | 67 | 167 | 176 | 160 |
| C | 52 | 159 | 151 | 148 | 161 | 151 | 175 | 166 | 161 | 175 | 148 | 166 |
| D | 52 | 149 | 163 | 154 | 160 | 180 | 165 | 168 | 163 | 173 | 156 | 158 |
| E | 43 | 155 | 169 | 191 | 168 | 160 | 166 | 163 | 182 | 202 | 166 | 157 |
| F | 44 | 170 | 155 | 168 | 161 | 170 | 161 | 161 | 161 | 173 | 165 | 163 |
| G | 52 | 167 | 121 | 159 | 172 | 163 | 161 | 154 | 183 | 124 | 162 | 155 |
| H | 52 | 148 | 163 | 163 | 165 | 156 | 165 | 162 | 163 | 167 | 166 | 163 |

Secondary confirmation assays are also performed. Hits are tested against the other anti-apoptotic members of the Bcl-2 family by FPA, determining the spectrum of activity of the compounds with respect to competitive binding for the pocket that binds BH3 peptides. To exclude compounds that non-specifically interfere with FPAs, compounds are tested in a FPA for another unrelated protein, which involves the BIR3 domain of XIAP binding to rhodamine-conjugated tetrapeptide AVPI, representing the N-terminus of the IAP antagonist SMAC (Liu et al., Nature 408:1004-1008 (2000); Wu et al., Nature 408:1008-1012 (2000)).

Cell-based assays are also used. A cell-based assay has previously been described where Bcl-X_{L} was co-expressed in HeLa cells with a GFP-tagged BH3 protein, and compounds were tested for their ability to displace the GFP-tagged BH3 protein from mitochondria-bound Bcl-X_{L} by confocal microscopy, using time-lapsed video microscopy (Becattini et al., Chem Biol 11:389-395 (2004); Leone et al., Cancer Res 63:8118-8121 (2003); Oltersdorf et al., Nature 435:677-681 (2005)). A similar cell line is engineered using Bfl-1 instead of Bcl-X_{L} and used as another secondary screen.

Another cell-based assay can also be used. Previously, stably transfected human cell lines were engineered to express Bcl-2 family members using a tetracycline-inducible promoter system. In these cells, turning on expression of anti-apoptotic Bcl-2-family member Bcl-X_{L} was shown to protect against apoptosis induced by cytotoxic anticancer drugs such as doxorubicin (Wang et al., J Biol Chem 279:48168-48176 (2004)). Addition of Bcl-X_{L} neutralizing compounds overcomes this protection. A tet-inducible HeLa cell line is similarly engineered to conditionally express Bfl-1. The ability of Bfl-1-selective compounds to overcome cytoprotection mediated by Bfl-1 with Bcl-X_{L} is compared. Selective compounds are expected to restore apoptosis sensitivity to Bfl-1-expressing but not Bcl-X_{L}-expressing HeLa cells.

Other types of assays can be used to test for the effect of compounds on biological activities. For example, compounds that neutralize Bcl-2 and Bcl-X_{L} have been used to study the effects in real-time of these anti-apoptotic proteins on Ca²⁺ regulation in cells (Palmer et al., Proc. Natl. Acad. Sci. USA 101:17404-17409 (2004)). These and other assays can be performed to test for the effect of a compound on a biological activity of an anti-apoptotic Bcl-2 polypeptide.

### Example 3

### Inhibitors of Anti-Apoptotic Bcl-2 Polypeptide Bfl-1

Compounds were screened for inhibitory activity of Bfl-1 essentially as described in Example 1. Compounds were screened from the MLSMR library for binding to Bfl-1 and other anti-apoptotic Bcl-2 family proteins and inhibitory activity. Exemplary inhibitory compounds are shown, e.g., in Table 6 below. Table 6 shows the structure, FPA dose response and TR-FRET dose response curves of exemplary inhibitory compounds. The FPA and TR-FRET assays were performed essentially as described in Example I. Table 6 also provides the formula, molecular weight, values for ClogP and polar surface area, and the number of H bond acceptors, H bond donors and rotatable bonds. The logP value of a compound, which is the logarithm of its partition coefficient between n-octanol and water log(coctanol/cwater), is a measure of the compound's hydrophilicity.

Several additional compounds were identified. From the initial screen, at least two core scaffold structures were identified. An exemplary sulfonyl pyrimidine core scaffold structure is shown below:

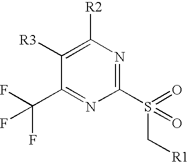

### Exemplary Sulfonyl Pyrimidine Core Scaffold

Shown in Table 7 below are exemplary analogs representative of a structure activity relationship (SAR) series. The activities of the compounds are also shown. An exemplary maleimide core scaffold structure is shown below:

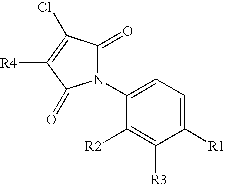

### Exemplary Maleimide Core Scaffold

Table 8 below shows exemplary analogs representative of a structure activity relationship (SAR) series. The activities of the compounds are also shown.

Additional data for compounds identified in the primary screening is available from the PubChem database (pubchem.ncbi.nlm.nih.gov). For example, a sulfonyl pyrimidine compound (compound 3 from Table 6) was tested in 123 additional assays, and demonstrated activity in fourteen of them, with seven of them being cellular toxicity assays. A maleimide compound (compound 12 from Table 6; see also Table 2) has demonstrated activity in 1 cytotoxicity assay and one more additional assay out of 77 screens.

### Example 4

### Evaluation of Commercially Available Chemical Inhibitors of Bcl-2 Polypeptides

This example illustrates the evaluation of (Z)-2-(5-(4-Bromobenzylidene)-4-oxo-2-thioxothiazolidin-3-yl)-3-methylbutanoic acid (BH31-1) and 3-chloro-1-(3,4-dichlorophenyl)-4-(dimethylamino)-1H-pyrrole-2,5-dione (CID-779754).

BH3I-1 is a commercially available small molecule inhibitor of the Bcl-2 family proteins. BH3I-1 possesses micromolar affinity for Bfl-1, Bcl-2, and Bcl-B, as shown in below, in addition to others as previously reported (Zhai et al., Cell Death and Differentiation 13, 1419-1421). In the FP assay described above, which was performed in the presence of 0.005% Tween 20, the affinity of the compound in Bfl-1 assay was significantly lower (IC50>>20 uM). Thus, BH3I-1 is at best a modestly potent, non-selective tool.

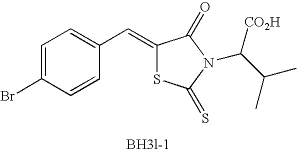

Bfl-1 (FP): IC₅₀=4.65 μM
Bcl-2 (FP): IC₅₀=1.14 μM
Bcl-B (FP): IC₅₀=1.08 μM

CID-779754 ((MLS-0067130), CID-779754, binds reversibly and competes with a peptide constituting the BH3 domain of Bid for binding to the Bfl-1 protein. Thus it is expected to potentially disrupt binding of other BH3-containing peptides and proteins with Bfl-1. CID-779754 displays weak activity in terms of competition for binding of a BH3 peptide to Bcl-2, suggesting that it may be selective for certain anti-apoptotic members of the Bcl-2 family (n=6 in humans).

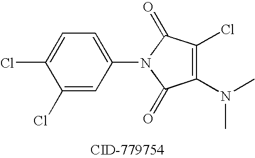

To validate the biological activity of the probe in a cellular readout that is Bfl-1 dependent, CID-779754 was tested in an assay measuring the effect of compounds on primary mast cell survival. In these studies it was found that CID-779754 inhibited the activation-induced survival of human mast cells, but that the compound is not toxic to normal human cells.

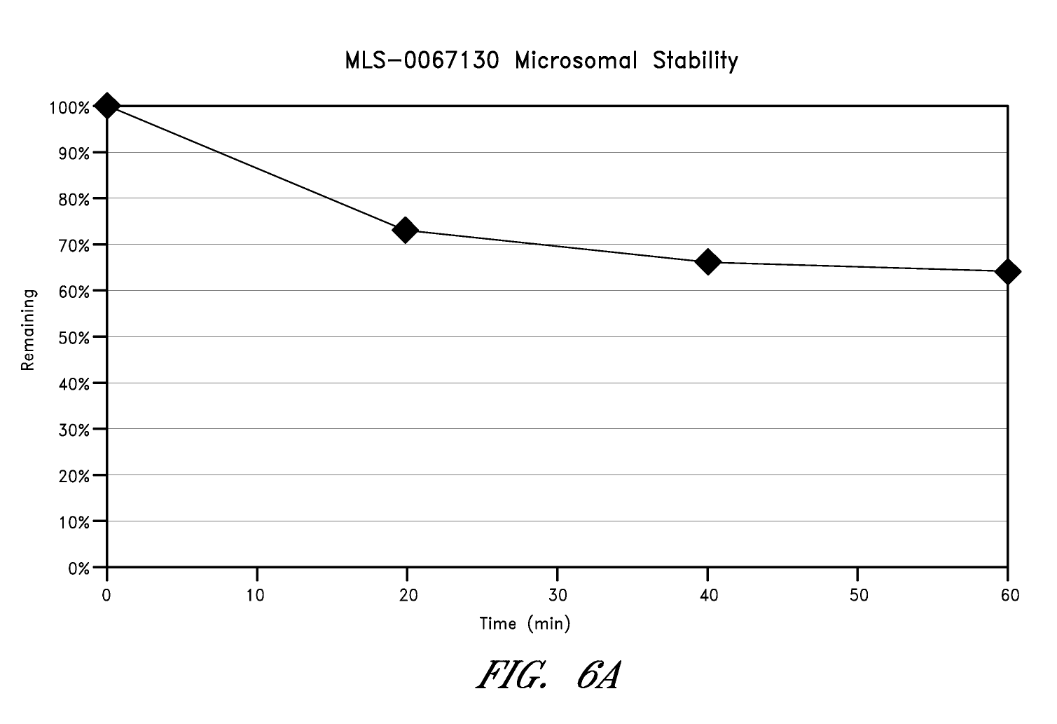

CID-779754 (MLS-0067130) has a metabolic half life of >60 min in the presence of rat liver microsomes when incubated at 37.5° C. (FIG. 6A), with 64% of the compound remaining at 1 hour. The plasma stability of CID-779754 is also good, with 79% remaining after incubating at 37.5° C. for 1 hour in fresh rat plasma (FIG. 6B). The compound shows high permeability in the PAMPA assay, with a log Pₑ of −4.2, which correlates to a predicted human GI absorption of >50%., showing t_{1/2} of >60 min.

CID-779754 demonstrated solubility at or below 100 uM in the biochemical assay. CID-779754 has some fluorescence in fluorescein channel at concentrations above 12.5 uM.

The structures and compound identifiers for commercially available chloromaleimide derivatives are shown in Table 2 below.

### Example 5

### Optimization of Chemical Inhibitors of Bfl-1

This example illustrates the optimization of chemical inhibitors of Bcl-2 polypeptides.

Compounds were screened for inhibitory activity of Bfl-1 essentially as described in Example 1, and including a 1 h incubation of Bfl-1 in the presence of compounds and a 4 h incubation after the subsequent addition of the fluorescent probe. The resulting assay was robust and characterized with a Z′-factor equal to 0.75. The assay was screened against the entire NIH small molecule library, which at the time consisted of 65,000 compounds. From this screen, 44 primary positives were obtained. The primary positives showed greater than 50% displacement in the FP assay and less than 50% increase in total fluorescence. IC₅₀ values in FP and TR-FRET assays were used to narrow the 44 primary positives to 20, then 14 candidates. Controls included an “artifact” plate on which a GAPDH assay was performed to check selectivity, and a solubility plate on which a solubility assay was performed to test solubility. Preferred compounds selected showed selectivity, solubility and a lack of artifacts. Less preferred compounds showed higher cross-reactivity and assay-interference.

The structure activity relationship (SAR) around 3-chloro-1-(3,4-dichlorophenyl)-4-(4-methylpiperazin-1-yl)pyrrole-2,5-dione (CID-1180676), which was identified as a Bfl-1 inhibitor, was initially investigated using three structural elements of CID-1180676: (a) the maleimide chloro substituent and carbon-carbon double bond; (b) the substituents on the phenyl ring; and (c) the piperazine moiety. The structure of CID-1180676 is shown below:

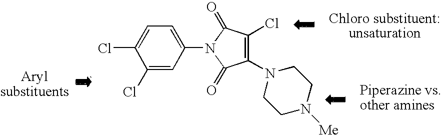

CID-1180676 was confirmed to possess submicromolar potency for inhibition of Bfl-1 in both the FP and TR-FRET assays (Table 3). It also appears to be highly selective, being inactive in 144 out of 151 assays tested as recorded in PubChem. Analogues of this compound were synthesized according to the synthetic pathway shown in scheme depicted below. In the depicted scheme, the reagents and conditions are as follows: (a) Dichloromaleic anhydride, AcOH, 110° C., 3 h; (b) (CH3)2NH, dioxane, 80° C., 20 h; 57% overall.

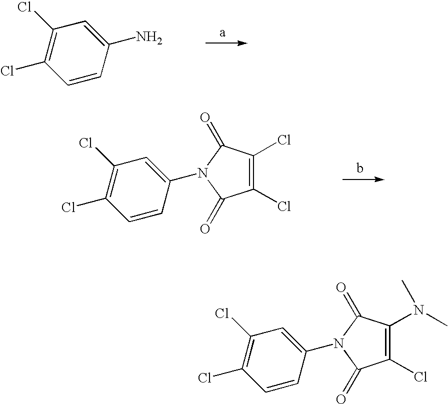

Substituted anilines are reacted with dichloromaleic anhydride in acetic acid at reflux to furnish the corresponding dichloromaleimides. Reaction of these products with various amines affords substitution of one of the chlorines to furnish the product aminomaleimides. Using this methodology, 280 analogues of CID-1180676 were prepared to optimize potency and selectivity for Bfl-1.

Two analogues, MLS-0066991 and MLS-0066987 were used to investigate the importance of the maleimide chloro substituent and carbon-carbon double bond. MLS-0066991 lacks both the halogen and the unsaturation, while MLS-0066987 lacks the halogen but retains the double bond. Both compounds were inactive in the FP and TR-FRET assays, suggesting that these structural elements are important for Bfl-1 activity. Mechanistic studies indicate that the chloroenone functionality present in the structure does not appear to make the molecule susceptible to irreversible covalent protein labeling. The structures of MLS-0066991 and MLS-0066987 are shown below:

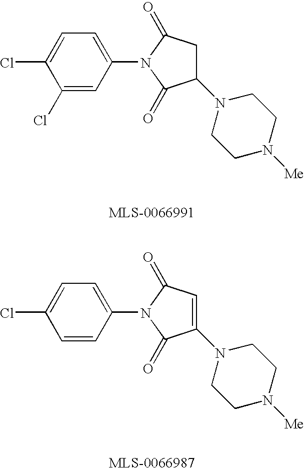

Using CID-1180676 as a template, a series of analogues was synthesized in which the substituents on the phenyl ring were varied. The Bfl-1 inhibitory activity of the analogues was evaluated using FP and TR-FRET assays. The results of this study are summarized in Table 3 below. With respect to the chloro substitution pattern, the 3,4-dichlorophenyl configuration present in CID-1180676 is preferred.

To investigate the SAR around the maleimide amine substituent, a series of analogues was prepared in which the piperazine moiety in CID-1180676 was replaced with different amines including dimethylamine, morpholine, piperidine, methoxyethylamine, pyrrolidine and aniline. The analogues in this series are shown in Table 4. The dimethylamine analogue (MLS-0067130) in this series was equipotent with CID-1180676 while the morpholine derivative (MLS-0067124), with IC₅₀ values of 1.4 μM and 1.14 μM in the FP and TR-FRET assays, respectively, was slightly less potent than CID-1180676. The other analogues in this series were less potent still, with the pyrrolidine and aniline derivatives being inactive at the doses tested.

A series of analogues were prepared in which the maleimide and phenyl portions of the molecule were retained intact while the piperazine moiety was modified with various substituents. The most active compounds generated are shown in Table 5 below.

The compounds synthesized include derivatives in which a variety of substituents are appended to the distal nitrogen of the piperazine moiety. In some compounds the substituents are lipophilic (as in MLS-0090859, MLS-0090860, MLS-0090866 and MLS-0090865) while other examples are more hydrophilic in nature (e.g. MLS-0090861, MLS-0090874 and MLS-0090868).

Other analogues, exemplified by MLS-0090857, MLS-0090858, and MLS-0090884, were modified at the level of the piperazine carbon framework. The analog MLS-0090861, contains a (2-hydroxyethoxy)ethyl substituent and has a slightly improved potency (IC₅₀=0.47 uM) and enhanced aqueous solubility compared with CID-1180676. Based on these studies, taken together with the mechanistic and confirmatory studies, the dimethylamine derivative MLS-0067130, derived from CID-1180676, was selected as a research probe for Bfl-1. MLS-0067130 has an IC₅₀ if 0.47 uM (TR-FRET).

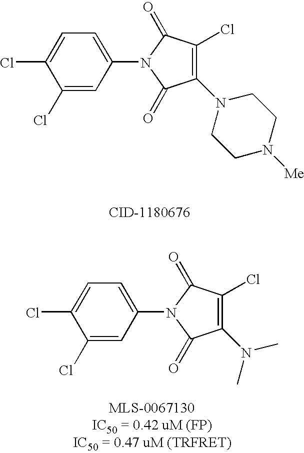

A detailed synthetic pathway for making the MLS-0067130 Bfl-1 probe is below. In the depicted scheme, the reagents and conditions are as follows: (a) Dichloromaleic anhydride, AcOH, 110° C., 3 h; (b) (CH₃)₂NH, dioxane, 80° C., 20 h; 57% overall.

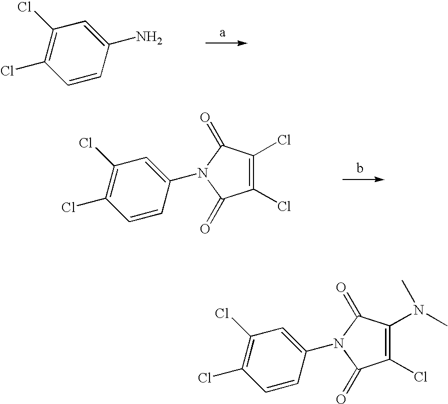

Probe PubChem Compound Identifier (CID/SID): CID-779754; SID-24810089, SID: 3332609, SID: 26514158, SID: 1008291, SID: 8214517, SID: 8043994, SID: 6898290, SID: 45153601, SID: 3962106; MLS-0067130 (maleimide).

### Example 6

### Selectivity Assay to Test Chemical Inhibitor Selectivity for Bcl-2 Family Members

This example illustrates assays for determining the selectivity of chemical inhibitors for Bfl-1. Bfl-1, as with other Bcl-2 family proteins, participates in a very complex and intricate network of interactions with other representatives of the family. Therefore, two selectivity assays involving family members Bcl-2 and Bcl-B were developed.

For Bcl-2, a FITC-conjugated peptide corresponding to the BH3 domain of Bim was used. For Bcl-B, a FITC-conjugated peptide corresponding to a fragment of the TR3/Nur77 protein shown to bind this member of the Bcl-2 family was used.

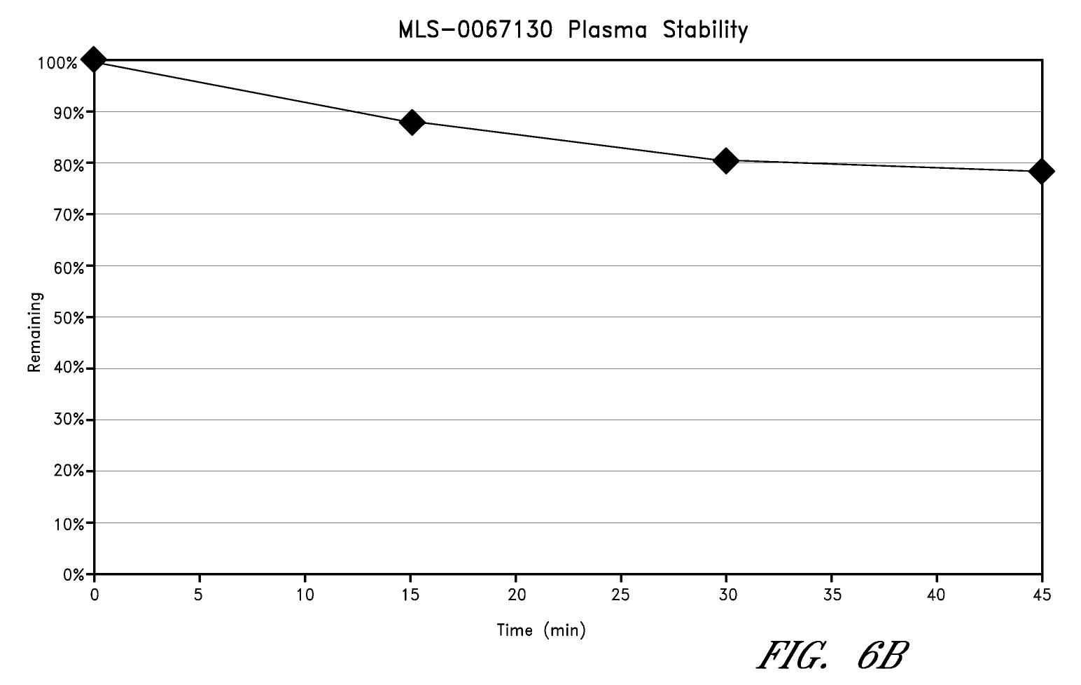

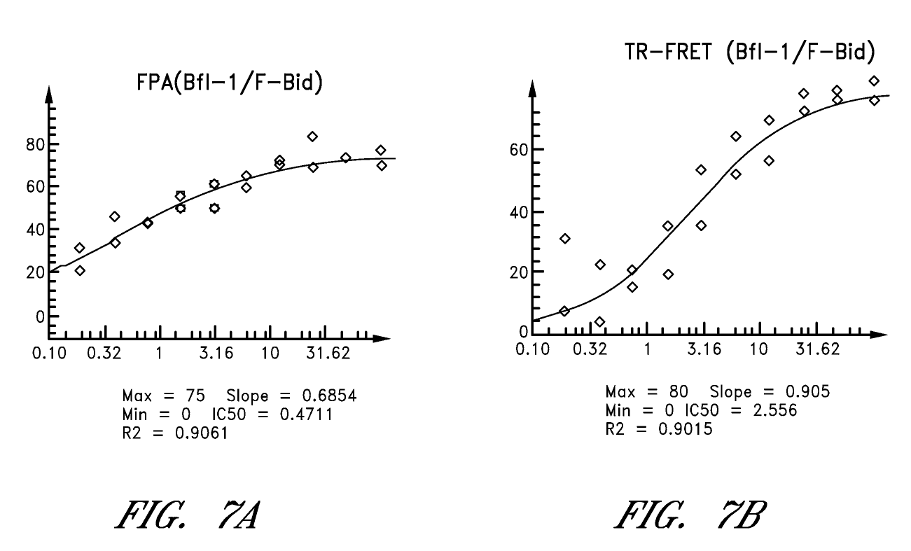

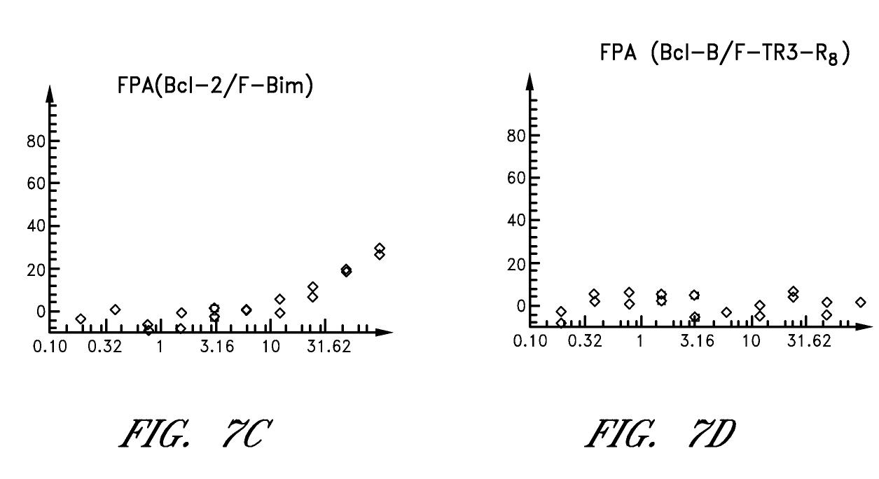

Probe specificity for target or for cell-based/phenotypic assays data can be determined in orthogonal cell-based assay systems that address the pathway of interest. For example, compounds were tested in Bfl-1/FITC-Bid FPA and TR-FRET assays (FIGS. 7A and 7B, respectively), as well as Bcl-2/FITC-Bim FPA (FIG. 7C) and Bcl-B/FITC-TR3-R₈ FPA (FIG. 7D) assays. Data represent concentration in μM x-axis) vs % displacement (y-axis). As shown in FIGS. 7A-7D, a chloromaleimide derivative (CID-1180676) was able to potently disrupt the complex Bfl-1/FITC-Bid (FIGS. 7A-7B), but showed limited activity against the Bcl-2/FITC-Bim complex (FIG. 7C) and did not affect the complex of Bcl-B/FITC-TR3-R₈ (FIG. 7D).

### Example 7

### Treatment of Cancer with a Chemical Inhibitor of Bfl-1

A patient diagnosed with a cancer tumor is selected for treatment with a chemical inhibitor of Bfl-1. The patient is given a therapeutically effective intravenous dose of the chemical inhibitor at regular intervals. At three, six, nine, and twelve weeks of treatment, the patient is evaluated by for shrinkage, growth or metastasis of the tumor. Following the end of the treatment period it is observed that the cancer has regressed.

### Example 8

### Treatment of Inflammation-Associated Disease with a Chemical Inhibitor of Bfl-1

A patient diagnosed with an inflammation-associated disease or autoimmunity-associated is selected for treatment with a chemical inhibitor of Bfl-1. The patient is given a therapeutically effective intravenous dose of the chemical inhibitor at regular intervals over a three, six, nine or twelve week period. Following the end of the treatment period it is observed that inflammation in the patient is reduced.

### Example 9

### Regulation of Activation-Induced Mast Cell Survival in Allergy

This Example illustrates one possible method of assessing induction of apoptosis in mast cells during allergic reaction by inhibiting the prosurvival protein Bfl-1. The sensitivity of primary mast cells to a small molecule inhibitor of Bfl-1 is also investigated. The effect of a Bfl-1 inhibitor on mast cell survival is determined as described below.

Mast cells are inflammatory cells of hematopoietic origin, distributed in almost all tissues in the body. Human mast cells are activated by cross-linkage of the high affinity IgE-receptor or ionomycin. Upon activation, mast cells release their granule content (e.g., histamine and proteases like tryptase, chymase and metalloproteinases), generate lipid mediators and secrete cytokines and growth factors. Although best known for their role in allergic reaction, mast cells are today considered to be versatile effector and regulatory cells, involved in many disorders such as autoimmunity. Benoist, C., and Mathis, D. 2002. Nature 420:875-878. More recent findings suggest also that mast cells are more central to both innate and acquired immune responses than previously believed. Galli et al. 2005. Nat Immunol 6:135-142. Mast cells are also believed to be a part of tumorigenesis in the development of several types of cancer. Thus, mast cells are important in several acute and chronic inflammatory diseases, both for the initiation as well as the perpetuation of the inflammation.

During allergic activation of mast cells (mice and human) through aggregation of the high-affinity IgE-receptor, the pro-survival gene A1/Bfl-1 is upregulated. Xiang, Z. et al., J. Exp. Med. 194:1561-1569; Xiang, Z. et al. 2006. Allergy 61:1040-1046. A1/Bfl-1 deficient mast cells do not exhibit such activation-induced mast cell. Xiang, Z. et al., J. Exp. Med. 194:1561-1569. Furthermore, sensitized A1 deficient mice show reduced active cutaneous anaphylaxis and reduced number of mast cells after provocation (Xiang et al. submitted).

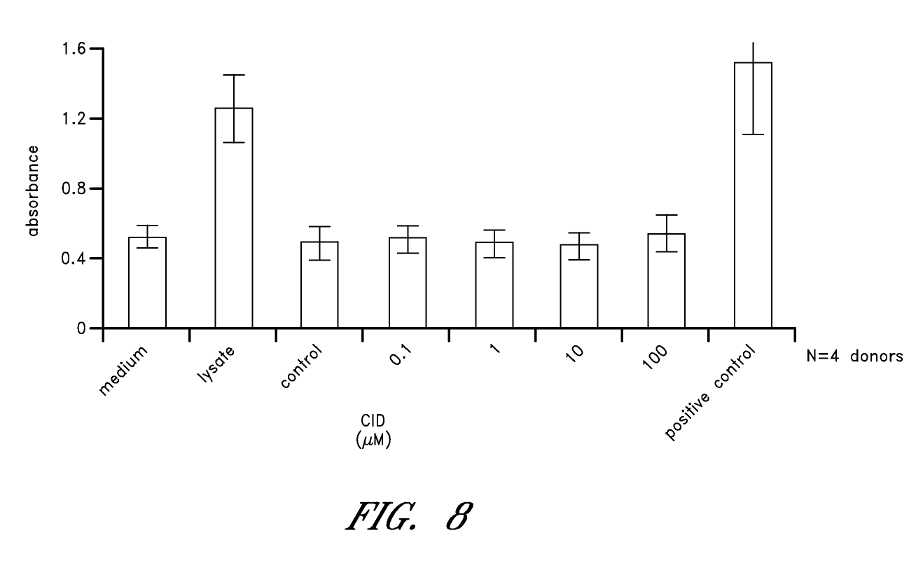

To investigate whether a Bfl-1 inhibitor is cytotoxic to cord blood derived human mast cells (CBMCs), a calorimetric assay based on the measurement of lactate dehydrogenase (LDH) activity is used. CBMC are cultured with various concentrations of the Bfl-1 inhibitor, and LDH release into the culture supernatant is measured. For example, FIG. 8 depicts results of an assay for CID-779754, which shows no cytoxicity at 0.1-100 μM (n=4 donors; mean±std dev). The positive control in FIG. 8 is a control included in the LDH kit. Thus, CID-779754 is not cytotoxic to in vitro developed human mast cells.

The effect of a Bfl-1 inhibitor at, for example, 1 μM is tested on CBMCs derived from, for example, 12 different individuals. CBMCs are activated by aggregation of the high-affinity IgE-receptor (FcεRI)±CID-779754. Mast cells are cultured with or without IgE crosslinking (IgECL) agent, without or with Bfl-1 inhibitor. The percentage cell survival upon IgE-receptor activation are compared to cells not activated and the delta survival is calculated. Cell survival is measured by LDH release and data were presented as % difference between cells cultured with or without IgECL.

### Example 10

### Induction of Apoptosis in Cancer Cells

This Example illustrates the induction of apoptosis in cancer cells using the compounds of the present invention.

To test compounds for their ability to induce apoptosis in cancer cells, the RS11846 B-lymphoma cell line was used. The RS11846 cell line was established from a patient with an aggressive form of diffuse large cell lymphoma, representing a subset of B-lymphoma in humans. This type of human B-lymphoma is characterized by infiltration beyond lymphoid compartment (bone marrow, spleen, lymph nodes, liver and etc.), typically infiltrate into ovary (in female), testis (in male), spinal cord and CNS (central nervous system) therefore given nomenclature of “diffuse.” Histo-pathologically, the RS11846 cell line is diffuse large cell B-lymphoma, thus representing an immature, aggressive form of the disease. When injected into SCID mice, RS11846 cells resulted in disseminated lymphoma and invariably resulted in spinal cord infiltration, and all mice developed “bilateral hind-leg paralysis” leading to death in approximately 4.5 weeks after inoculation via intraperitoneal (i.p.) or intravenous (i.v.). Karyotype analysis of the RS11846 cell line revealed a t14:18 chromosomal translocation, thereby resulting in overexpression of Bcl-2 family proteins, including overexpression of Bfl-1. Therefore, this cell line is suitable for examining biological effects of Bfl-1 antagonists concerned in this patent application.

For the assay to test compounds for their ability to induce apoptosis, the RS11846 B-lymphoma cell line was cultured in RPMI-1640 supplemented by 10% FBS and Penicillin/Streptomycin. The cells were seeded at cell density of half a million cells per ml, and incubation time was 16 hours. In the assay, cell viability was analyzed by the Annexin-FITC/PI method, using flow cytometry, or FACS analysis. Viable cells were defined by Annexin-FITC-negative, PI-negative. Compensation was done using conventional methods and using the Flo-jo program.

IC₅₀ (50% killing) was determined using an extrapolation method based on the finding that biological response is almost linear near at 50% inhibition point when plotted against semi-log scale on X-axis. See, D. J. Finney, Statistical Method in Biological Assay, Griffin, London, 1978. Briefly, % viability was plotted on Y-axis on an ordinary scale, while drug concentration was plotted on X-axis on a semi-log scale. The actual 50% inhibition point is on the line between left-bracket (higher than 50% inhibition) and right bracket (lower than 50% inhibition), and it can be extrapolated on the line on a semi-log scale. Thus, IC₅₀ was extrapolated via left bracketing and right bracketing, and extrapolated on a semi-log scale, and then, re-converted back to an ordinary scale.

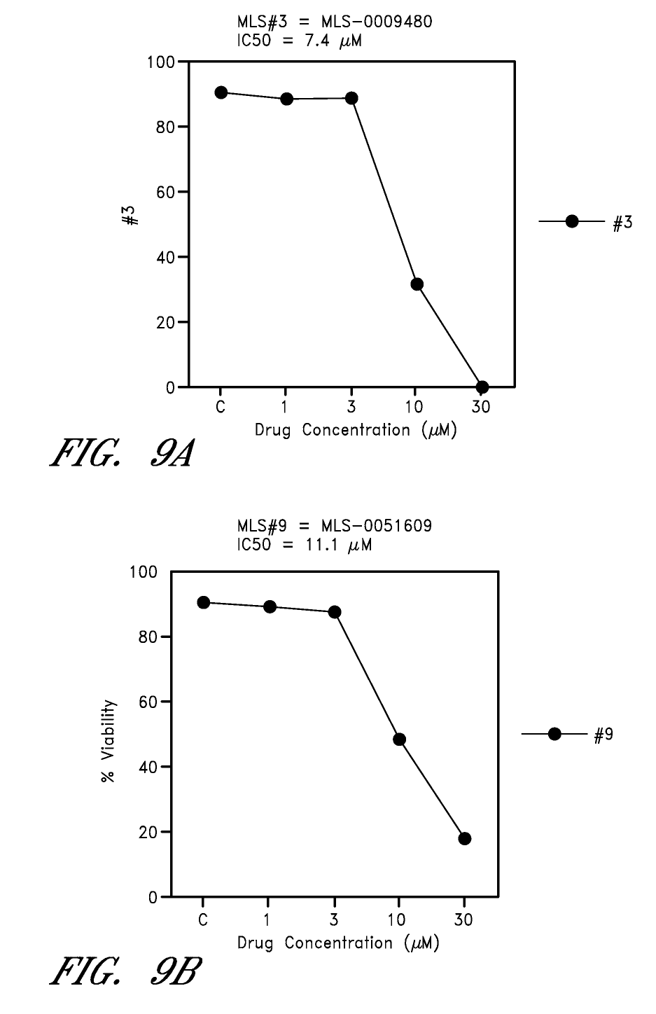

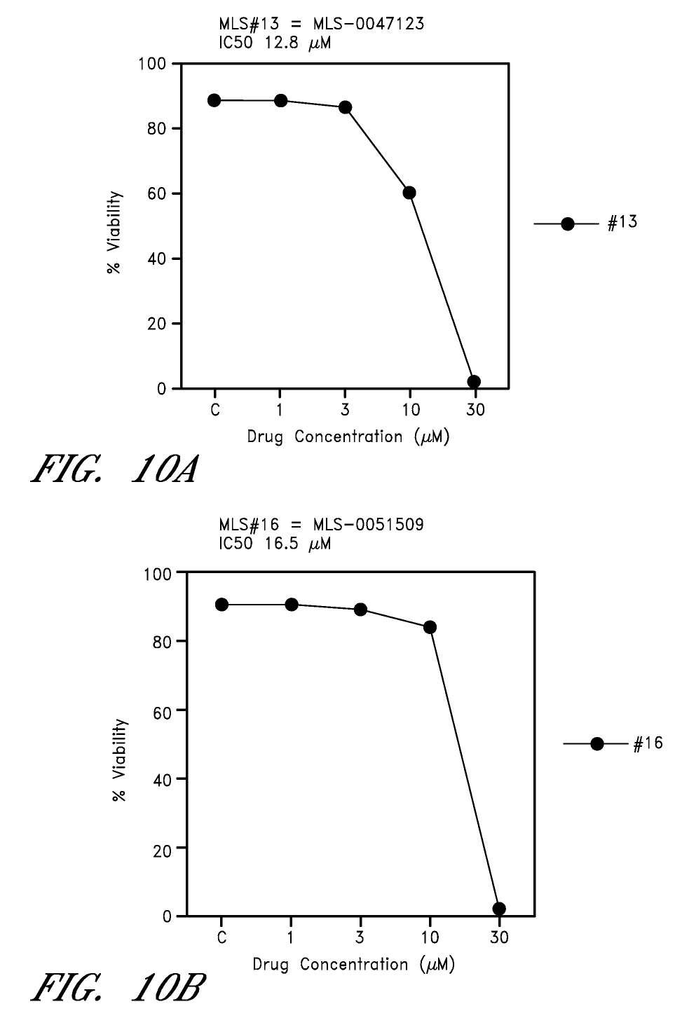

Of a total of 21 MLS-compounds examined, only 4 compounds induced apoptosis in a dose-dependent manner in RS11846 lymphoma cell line, IC₅₀ ranging from 7.4 μM to 16.5 μM, somewhat higher than IC₅₀ for FPA and FRET. See, FIGS. 9 and 10. Surprisingly, many MLS-compounds did not induce apoptosis.

While the present teachings have been described in terms of these exemplary embodiments, the skilled artisan will readily understand that numerous variations and modifications of these exemplary embodiments are possible without undue experimentation. All such variations and modifications are within the scope of the current teachings.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

TABLE 2
Structures and compound identifiers for commercially available
chloromaleimide derivatives

| CID # | Structure | SID # | Vendor | Cat. No. | MLS # |
|---|---|---|---|---|---|
| 1180676 | 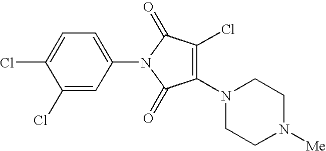 | 26514112 | Chembridge | 6500959 | 0053105 |
| 779754 | 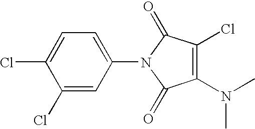 | 3962106 | Chembridge | 5354778 | 0067130 |
| 1180675 | 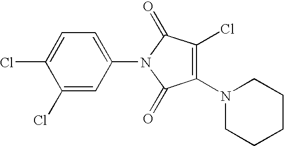 | 26514119 | Chembridge | 5344200 | 0066990 |

TABLE 3
SAR for substituents around phenyl ring.

| | | Bfl-1 FP | Bfl-1 TR FRET |
|---|---|---|---|
| Compound # | Structure | IC₅₀ (uM) | IC₅₀ (uM) |
| CID-1180676 | 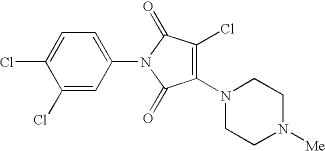 | 0.75 | 0.56 |
| MLS-0090834 | 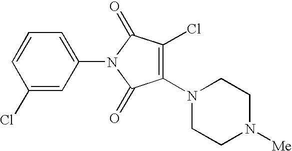 | 2.15 | 1.17 |
| MLS-0090829 | 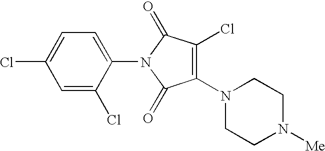 | 2.62 | 1.36 |
| MLS-0090833 | 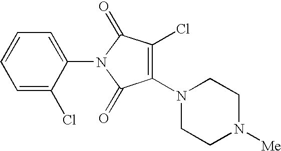 | 2.16 | 1.45 |
| MLS-0090828 | 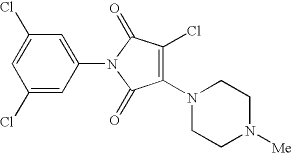 | 2.44 | 1.66 |
| MLS-0090831 | 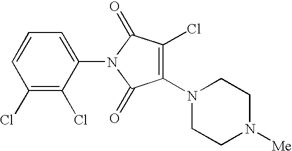 | 8.74 | 2.56 |
| MLS-0090830 | 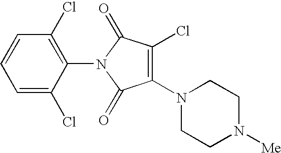 | 13.74 | 3.72 |
| MLS-0090832 | 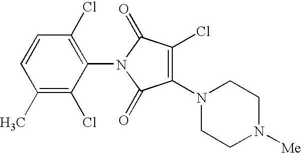 | 23.9 | 11.1 |

TABLE 4
SAR for amine substituents on the maleimide ring.

| | | Bfl-1 FP | Bfl-1 TR FRET |
|---|---|---|---|
| Compound # | Structure | IC₅₀ (uM) | IC₅₀ (uM) |
| CID-1180676 | 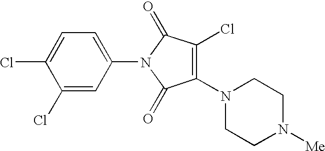 | 0.75 | 0.56 |
| MLS-0067130 | 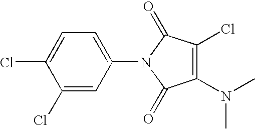 | 0.46 | 0.57 |
| MLS-0067124 | 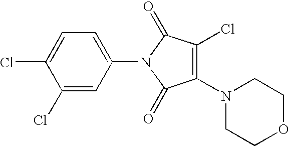 | 1.41 | 1.14 |
| MLS-0066990 | 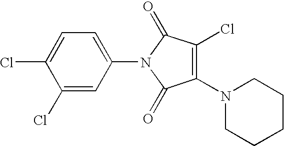 | no fit | 1.3 |
| MLS-0067127 | 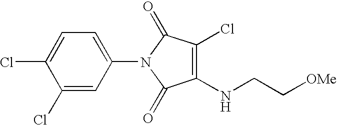 | 12.6 | 4.96 |
| MLS-0067126 | 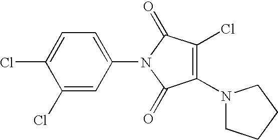 | no fit | no fit |
| MLS-0067128 | 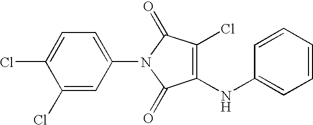 | no fit | no fit |

TABLE 5
SAR for derivatives in which the piperazine has been modified.

| | | Bfl-1 FP | Bfl-1 TR FRET |
|---|---|---|---|
| Compound # | Structure | IC₅₀ (uM) | IC₅₀ (uM) |
| MLS-0090861[ [ | 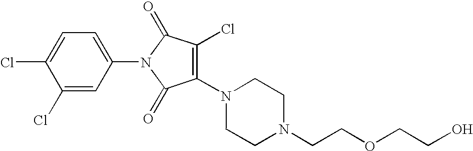 | 0.47 | 0.42 |
| MLS-0090874 | 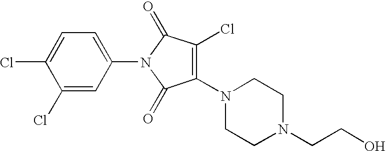 | 0.56 | 0.59 |
| MLS-0090859 | 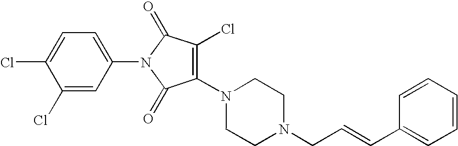 | 0.59 | 0.73 |
| MLS-0090857 | 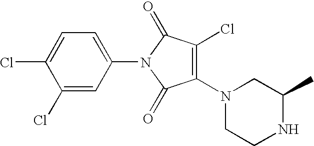 | 1.16 | 1.03 |
| MLS-0090860 | 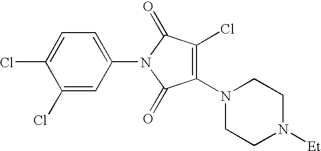 | 1.67 | 0.97 |
| MLS-0090858 | 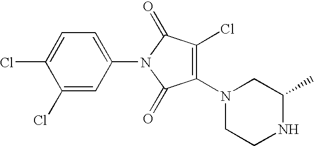 | 1.32 | 1.25 |
| MLS-0090866 | 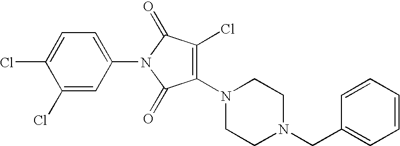 | 1.62 | 1.64 |
| MLS-0090868 | 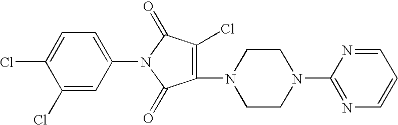 | 1.9 | 2.46 |
| MLS-0090884 | 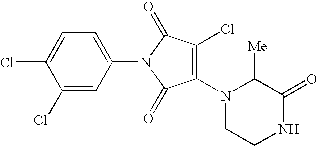 | 5.63 | 2.03 |
| MLS-0090865 | 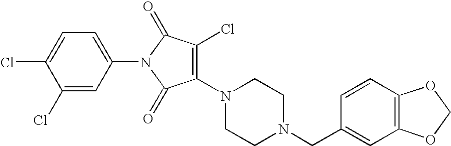 | 4.07 | 2.14 |

TABLE 6
Exemplary anti-apoptotic Bcl-2 polypeptide inhibitory compounds.

| Com- | | Substance | | |
|---|---|---|---|---|
| pound | Structure | ID | FPA dose-response | TR-FRET dose response |
| 1 | 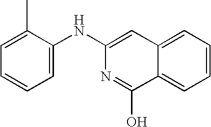 | MLS-0004065 | 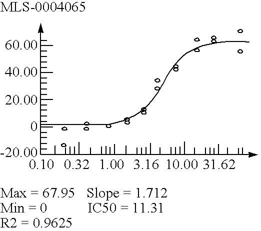 | 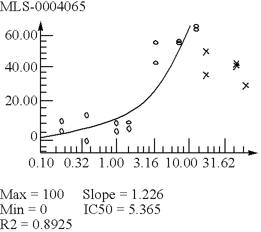 |

| Formula | Mol Wt | ClogP | Polar Surface Area | H Bond Acceptor | H Bond Donor | Rotatable Bond |
|---|---|---|---|---|---|---|
| C₁₆H₁₄N₂O | 250.3 | 4.46 | 45.15 | 3 | 2 | 2 |

| Com- | | Substance | | |
|---|---|---|---|---|
| pound | Structure | ID | FPA dose-response | TR-FRET dose response |
| 2 | 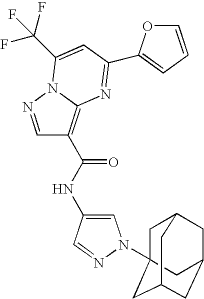 | MLS-008158 | 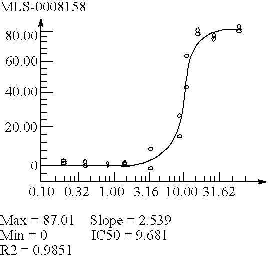 | 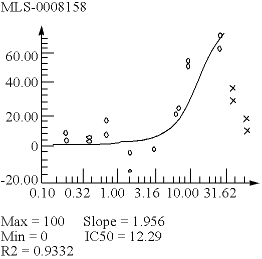 |

| Formula | Mol Wt | ClogP | Polar Surface Area | H Bond Acceptor | H Bond Donor | Rotatable Bond |
|---|---|---|---|---|---|---|
| C₂₅H₂₃F₃N₆O₂ | 496.48 | 4.393 | 90.25 | 4 | 1 | 5 |

| Com- | | Substance | | |
|---|---|---|---|---|
| pound | Structure | ID | FPA dose-response | TR-FRET dose response |
| 3 | 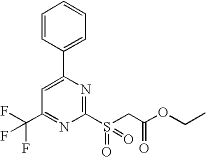 | MLS-009480 | 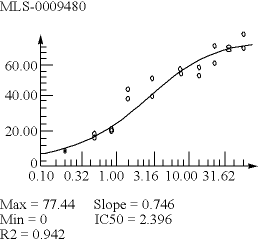 | 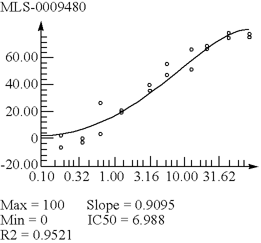 |

| Formula | Mol Wt | ClogP | Polar Surface Area | H Bond Acceptor | H Bond Donor | Rotatable Bond |
|---|---|---|---|---|---|---|
| C₁₅H₁₃F₃N₂O₄S | 374.33 | 3.471 | 94.6 | 6 | 0 | 7 |

| Compound | Structure | Substance ID |
|---|---|---|
| 4 | 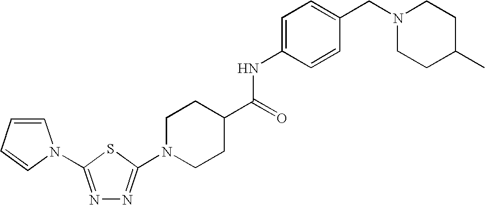 | MLS-0012219 |

| Compound | FPA dose-response | TR-FRET dose response |
|---|---|---|
| 4 | 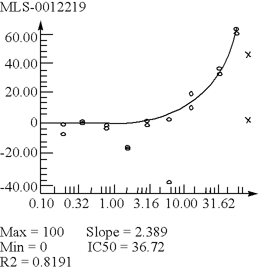 | 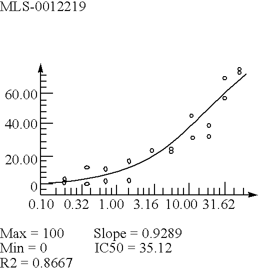 |

| Formula | Mol Wt | ClogP | Polar Surface Area | H Bond Acceptor | H Bond Donor | Rotatable Bond |
|---|---|---|---|---|---|---|
| C₂₅H₃₂N₆OS | 464.63 | 3.763 | 94.53 | 5 | 1 | 6 |

| Com- | | Substance | | |
|---|---|---|---|---|
| pound | Structure | ID | FPA dose-response | TR-FRET dose response |
| 5 | 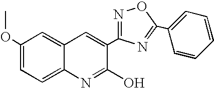 | MLS-0019296 | 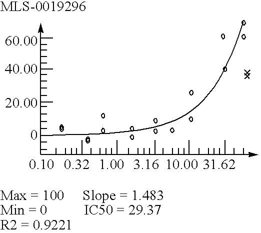 | 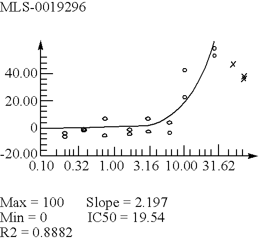 |

| Formula | Mol Wt | ClogP | Polar Surface Area | H Bond Acceptor | H Bond Donor | Rotatable Bond |
|---|---|---|---|---|---|---|
| C₁₈H₁₃N₃O₃ | 319.31 | 3.612 | 81.27 | 5 | 1 | 3 |

| Com- | | Substance | | |
|---|---|---|---|---|
| pound | Structure | ID | FPA dose-response | TR-FRET dose response |
| 6 | 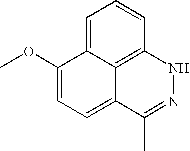 | MLS-0020314 | 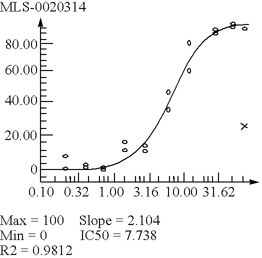 | 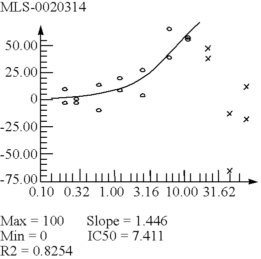 |

| Formula | Mol Wt | ClogP | Polar Surface Area | H Bond Acceptor | H Bond Donor | Rotatable Bond |
|---|---|---|---|---|---|---|
| C₁₃H₁₂N₂O | 212.25 | 2.491 | 33.61 | 3 | 1 | 1 |

| Compound | Structure | Substance ID |
|---|---|---|
| 7 | 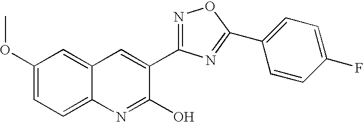 | MLS-0025736 |

| Compound | FPA dose-response | TR-FRET dose response |
|---|---|---|
| 7 | 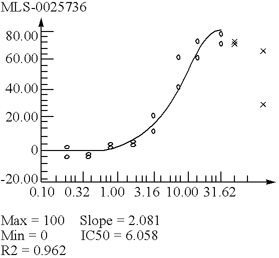 | 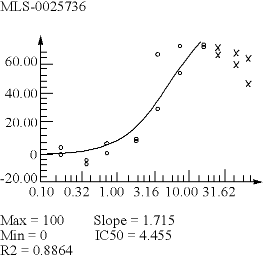 |

| Formula | Mol Wt | ClogP | Polar Surface Area | H Bond Acceptor | H Bond Donor | Rotatable Bond |
|---|---|---|---|---|---|---|
| C₁₈H₁₂FN₃O₃ | 337.3 | 3.817 | 81.27 | 5 | 1 | 3 |

| Com- | | Substance | | |
|---|---|---|---|---|
| pound | Structure | ID | FPA dose-response | TR-FRET dose response |
| 8 | 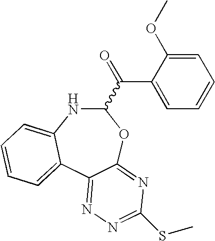 | MLS-0046088 | 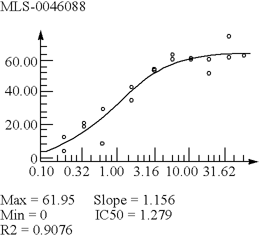 | 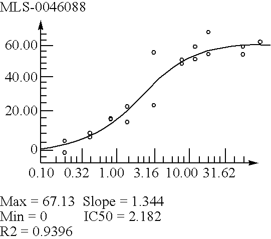 |

| Formula | Mol Wt | ClogP | Polar Surface Area | H Bond Acceptor | H Bond Donor | Rotatable Bond |
|---|---|---|---|---|---|---|
| C₁₉H₁₆N₄O₃S | 380.42 | 4.401 | 111.53 | 8 | 1 | 4 |

| Com- | | Substance | | |
|---|---|---|---|---|
| pound | Structure | ID | FPA dose-response | TR-FRET dose response |
| 9 | 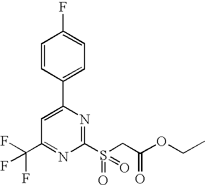 | MLS-0047123 | 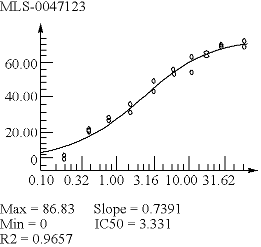 | 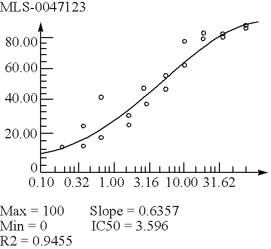 |

| Formula | Mol Wt | ClogP | Polar Surface Area | H Bond Acceptor | H Bond Donor | Rotatable Bond |
|---|---|---|---|---|---|---|
| C₁₅H₁₂F₄N₂O₄S | 392.33 | 3.677 | 94.6 | 6 | 0 | 7 |

| Compound | Structure | Substance ID |
|---|---|---|
| 10 | 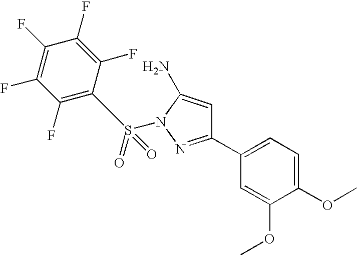 | MLS-0051509 |

| Compound | FPA dose-response | TR-FRET dose response |
|---|---|---|
| 10 | 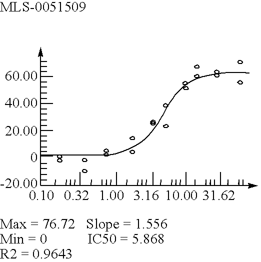 | 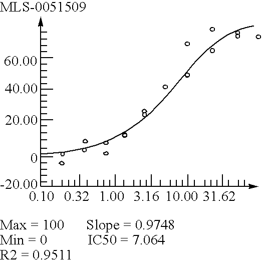 |

| Formula | Mol Wt | ClogP | Polar Surface Area | H Bond Acceptor | H Bond Donor | Rotatable Bond |
|---|---|---|---|---|---|---|
| C₁₇H₁₂F₅N₃O₄S | 449.35 | 3.95 | 104.82 | 6 | 1 | 5 |

| Com- | | Substance | | |
|---|---|---|---|---|
| pound | Structure | ID | FPA dose-response | TR-FRET dose response |
| 11 | 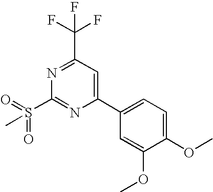 | MLS-0051609 | 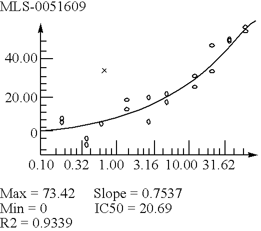 | 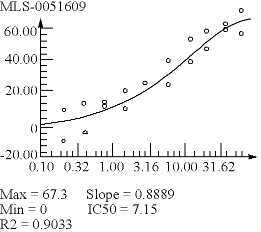 |

| Formula | Mol Wt | ClogP | Polar Surface Area | H Bond Acceptor | H Bond Donor | Rotatable Bond |
|---|---|---|---|---|---|---|
| C₁₄H₁₃F₃N₂O₄S | 362.32 | 3.383 | 86.76 | 6 | 0 | 6 |

| Com- | | Substance | | |
|---|---|---|---|---|
| pound | Structure | ID | FPA dose-response | TR-FRET dose response |
| 12 | 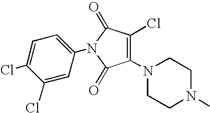 | MLS-0053105 | 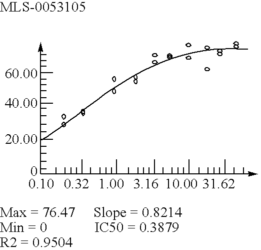 | 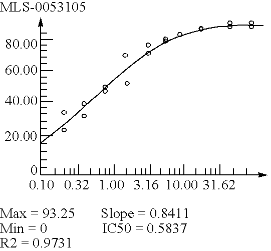 |

| Formula | Mol Wt | ClogP | Polar Surface Area | H Bond Acceptor | H Bond Donor | Rotatable Bond |
|---|---|---|---|---|---|---|
| C₁₅H₁₄C₁₃N₃O₂ | 374.65 | 2.814 | 43.86 | 4 | 0 | 2 |

TABLE 7
Exemplary sulfonyl pyrimidine core scaffold structure and
exemplary analogs representative of a structure activity relationship (SAR) series.

| | | | | | | | | Bfl-1 TR- | |
|---|---|---|---|---|---|---|---|---|---|
| | | | | | | | Bfl-1 FPA | FRET IC₅₀ | PubChem_{—} |
| Vendor | Cat # | R1 | R2 | R3 | Mol Wt | ClogP | IC50 (uM) | (uM) | SID |
| ChemDiv | C071-0649 | 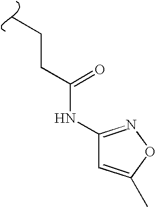 | 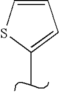 | H | 460.4 | 3.7 | 40.4 | 11.4 | 4252027 |
| ChemDiv | C164-0003 | H | 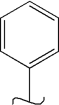 | H | 302.3 | 3.4 | nodisplacement | nodisplacement | 4241889 |
| ChemBridge | 7990929 | 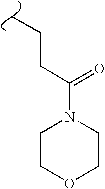 | 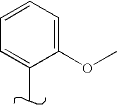 | H | 473.5 | 2.9 | 21.5 | 19.1 | 4264384 |
| ChemBridge | 7979975 | 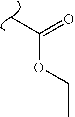 | 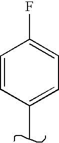 | H | 392.3 | 3.7 | 1.98/1.27/0.695/1.72/0.866/3.33/4.4/6.43/Avg 2.59 | 1.37/0.59/1.5/2.79/1.03/3.41/Avg 1.78 | |
| ChemDiv | C071-0367 | 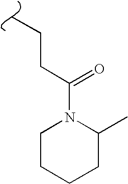 | 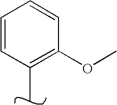 | H | 485.5 | 4.5 | 14.7 | 12.8 | 4243886 |
| ChemDiv | C164-0015 | H | 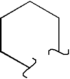 | 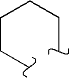 | 280.3 | 3.0 | nodisplacement | nodisplacement | 4248026 |
| ChemBridge | 7663066 | 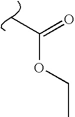 | 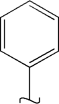 | H | 374.3 | 3.5 | 2.63/2.4/5.42/8.1/Avg 4.64 | 1.18/4.63/Avg 2.91 | |
| ChemBridge | 7914223 | 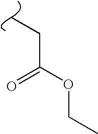 | 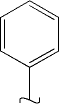 | H | 388.4 | 3.7 | 7.9 | 4.0 | 4263828 |
| ChemDiv | C071-0355 | 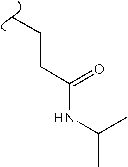 | 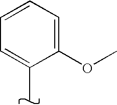 | H | 445.5 | 3.8 | nodisplacement | nodisplacement | 4243278 |
| ChemDiv | C164-0005 | H | 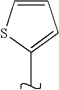 | H | 308.3 | 3.4 | 6.3 | 15.6 | 4251594 |
| AsinexLtd. | BAS03664947 | 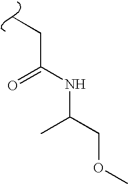 | 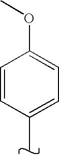 | H | 461.5 | 3.0 | 20.6 | 19.6 | 852084 |
| AsinexLtd. | ASN05990586 | 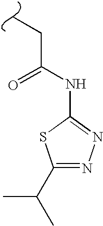 | 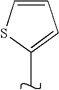 | H | 491.5 | 4.1 | 11.3 | 5.3 | 852701 |
| ChemDiv | C164-0006 | H | 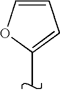 | H | 292.2 | 2.8 | 7.2 | 6.0 | 4243100 |
| ChemDiv | C164-0014 | H | 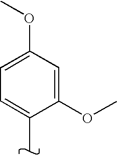 | H | 362.3 | 3.4 | 40.7/5.92/27.2/28.8/22.9/Avg25.1 | 5.29/3.73/10.1/Avg 6.38 | |
| ChemDiv | C164-0017 | H | 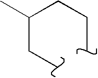 | 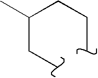 | 294.3 | 3.2 | nodisplacement | nodisplacement | 4252269 |
| ChemBridge | 7979496 | 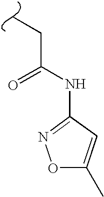 | 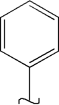 | H | 440.4 | 3.4 | 14.6 | 9.2 | 4254739 |
| ChemDiv | C071-0415 | 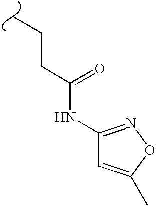 | 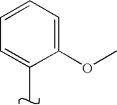 | H | 484.4 | 3.7 | nodisplacement | nodisplacement | 4244703 |

TABLE 8
Exemplary analogs representative of a structure activity relationship (SAR) series.

| | | | | | | | | Bfl-1 TR- | | Pub |
|---|---|---|---|---|---|---|---|---|---|---|
| | | | | | | | Bfl-1 FPA | FRET IC₅₀ | | Chem_{—} |
| Vendor | Cat # | R1 | R2 | R3 | R4 | Mol Wt | IC₅₀ (uM) | (uM) | ClogP | SID |
| Chem Div | 3453-0809 | 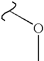 | H | H | 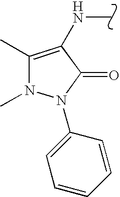 | 438.9 | 82.2 | >100.0 | 1.65 | 4247038 |
| ChemBridge | 5343472 | H | Cl [symbol] | H | 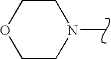 | 327.2 | >10.0 | 5.8 | 1.88 | |
| ChemBridge | 5346071 | H | H | Cl [symbol] | 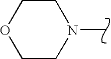 | 327.2 | 8.5 | 6.4 | 1.88 | |
| ChemBridge | 6500959 | Cl [symbol] | H | Cl [symbol] | 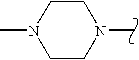 | 374.6 | 0.632/0.183/0.336/2.81/0.534/0.388/0.928/0.19/0.335/0.273/0.581/0.216/Avg 0.617 | 0.317/0.471/0.445/1.09/0.475/0.584/0.186/nofit/0.304/0.443/Avg0.479 | 2.81 | |

[symbol]

indicates data missing or illegible when filed

What is claimed is:

1. A method of modulating the activity of Bfl-1, comprising contacting a Bfl-1 polypeptide with a small molecule.

2. The method of claim 1, wherein said small molecule is selected from the group of molecules shown in Tables 2-8.

3. The method of claim 1, wherein said small molecule has a core structure selected from core structures I, II, III and IV.

4. The method of claim 1, wherein said small molecule is a derivative of 3-chloro-1-(3,4-dichlorophenyl)-4-(4-methylpiperazin-1-yl)pyrrole-2,5-dione.

5. The method of claim 4, wherein said small molecule has the following structure:

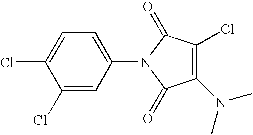

6. The method of claim 1, wherein said activity of Bfl-1 is binding of the Bfl-1 polypeptide to a BH3 domain.

7. The method of claim 6, wherein the binding of the Bfl-1 polypeptide to a BH3 domain is inhibited.

8. The method of claim 1, wherein said contacting occurs in a cell.

9. The method of claim 8, wherein the cell is a cancer cell.

10. The method of claim 1, wherein the small molecule is selected from the group consisting of MLS-0009480, MLS-0051609, MLS-0047123 and MLS-0051509.

11. A method of increasing apoptosis in a cell, comprising contacting a cell with an effective amount of a compound having a core structure selected from core structures I, II, III and IV or a compound selected from the compounds shown in Tables 2-8, whereby binding of a Bfl-1 polypeptide to a BH3 domain is inhibited and apoptosis is increased.

12. The method of claim 11, wherein the cell is a cancer cell.

13. The method of claim 11, wherein the compound is selected from the group consisting of MLS-0009480, MLS-0051609, MLS-0047123 and MLS-0051509.

14. The method of claim 11, wherein the compound has an IC₅₀ ranging from about 7.4 μM to about 16.5 μM.

15. A method of reducing the severity of a pathological condition in an individual, comprising administering to an individual having a pathological condition characterized by a pathologically reduced level of apoptosis a compound having a core structure selected from core structures I, II, III and IV or a compound selected from the compounds shown in Tables 2-8, whereby binding of a Bfl-1 polypeptide to a BH3 domain is inhibited and the severity of said pathological condition is reduced.

16. The method of claim 15, wherein said pathological condition is cancer.

17. The method of claim 15, wherein said pathological condition is selected from psoriasis, hyperplasia, an autoimmune disease, an inflammation-associated disease and restenosis.

18. The method of claim 15, further comprising administering a second therapeutic agent.

19. The method of claim 18, wherein the second therapeutic agent is selected from the group consisting of an alkylating agent, an antimetabolite, an antibody, a plant alkaloid, an antibiotic, an inorganic ion, a biological response modifier, and a hormone.

20. The method of claim 15, wherein the compound is selected from the group consisting of MLS-0009480, MLS-0051609, MLS-0047123 and MLS-0051509.

21. A method of screening for compounds capable modulating the activity of Bfl-1, comprising:
providing a Bfl-1 polypeptide;
providing a fluorescently labeled compound known to bind Bfl-1; and
contacting said Bfl-1 polypeptide and said binding compound in the presence or absence of a candidate binding compound or library of candidate binding compounds; and
determining fluorescence of said Bfl-1 polypeptide, wherein a decrease in fluorescence indicates that said candidate binding compound inhibits binding of said binding compound to said Bfl-1 polypeptide.

22. The method of claim 21, wherein said compound known to bind to Bfl-1 is selected from the group consisting of a peptide, peptide analog and small molecule.

23. The method of claim 22, wherein said peptide is Bid-BH3 peptide.

24. The method of claim 21, wherein said Bfl-1 polypeptide lacks a C-terminal hydrophobic transmembrane domain.

25. The method of claim 21, further comprising at least one secondary screen to confirm that said candidate binding compound modulates the activity of Bfl-1.


SEQUENCE LISTING (1 sequence)

SEQ ID NO: 1 - protein, 20 aa, Artificial Sequence
  Synthetic polypeptide
EDIIRNIARHLAQVGDSMDR